(19) United States
(12) Patent Application Publication
(10) Pub. No.: US 2015/0374237 A1
(43) Pub. Date: Dec. 31, 2015

(54) METHOD FOR ACCURATE AND ROBUST CARDIAC MOTION SELF-GATING IN MAGNETIC RESONANCE IMAGING

(71) Applicant: THE REGENTS OF THE UNIVERSITY OF CALIFORNIA, Oakland, CA (US)

(72) Inventors: Peng HU, Los Angeles, CA (US); Fei HAN, Los Angeles, CA (US); Stanislas RAPACCHI, Los Angeles, CA (US)

(21) Appl. No.: 14/764,972

(22) PCT Filed: Jan. 30, 2014
(86) PCT No.: PCT/US2014/013904

§ 371 Date: Jul. 30, 2015

Related U.S. Application Data
(60) Provisional application No. 61/759,379, filed on Jan. 31, 2013.

(51) Int. Cl. A61B 5/00; A61B 5/055

(52) U.S. Cl. CPC A61B 5/0044; A61B 5/055; A61B 5/7267; A61B 5/7285

(57) ABSTRACT

Self-gating methods and Systems are provided for cardiac imaging analysis. In particular, non-phased coded self-gating data are collected separately from imaging data. The method uses multiple coil arrays to repeatedly acquire self-gating signals that are separate from image acquisitions. Learning-based algorithms are used in data processing to detect a triggering event, such as the onset of a heartbeat.

### CROSS-REFERENCE OF RELATED APPLICATION

This application claims priority to U.S. Provisional Application No. 61/759,379, filed on Jan. 31, 2013 and entitled “Method For Accurate And Robust Cardiac Motion Self-Gating In Magnetic Resonance Imaging,” which is hereby incorporated by reference herein in its entirety.

### STATEMENT REGARDING FEDERALLY-SPONSORED RESEARCH

This invention was made with Government support under Grant No. HL113427, awarded by the National Institutes of Health. The Government has certain rights in this invention.

### FIELD

The invention disclosed herein generally relates to methods for data collection and signal processing. In particular, the invention disclosed herein generally relates to methods and systems of self-gating to provide synchronization signal to the imaging system

### BACKGROUND

In cardiac Magnetic Resonance Imaging (MRI) applications, electrocardiograph (ECG) is usually used to monitor the cardiac motion and provide synchronization (gating) signal to the imaging system. Although ECG-Gating is considered the clinical standard for cardiac MRI, it is still problematic in several aspects. First, the ECG signal is often interfered by the potent and fast varying magnetic field of the MRI scanner. Such interference could potentially cause inaccurate or even failed synchronization, leading to an unsuccessful imaging. Second, in clinical cardiac MRI protocols, additional time is required to set-up the ECG monitoring system prior to the imaging process. Sometimes, this process has to be repeated for a reliable ECG signal. Since the cost of a single MRI scan is directly related to the time required at the scanner, the need of ECG increases the cost of cardiac MRI scans, making the cardiac MRI one of the most expensive MRI scan process. Thirdly, ECG could be unstable for some individual patient (e.g., patient with hairy chest or abnormal chest and cardiovascular geometry) and even inaccessible for some special applications (e.g., fetus cardiac scan).

What is needed in the art are methods and systems for overcoming the aforementioned disadvantages of ECG-Gating. In particular, what is needed are improvements existing ECG-Gating technologies or alternatives/replacements thereof.

### SUMMARY

Provided herein is method for synchronizing image data acquisition during Magnetic Resonance Imaging (MRI). The method comprises a step of acquiring a self-gating dataset comprising a first plurality of subsets of self-gating data of the center k-space entire line, wherein the self-gating data are acquired separately from any imaging data, and wherein the first plurality of subsets of self-gating data is collected during the same cardiac cycle.

In some embodiments, the self-gating data is acquired using a plurality of radio frequency (RF) coil arrays. In some embodiments, the first plurality of subsets of self-gating data is non-phase encoded.

In some embodiments, the self-gating dataset further comprises a second plurality of subsets of self-gating data.

In some embodiments, the first plurality and second plurality of subsets of self-gating data are collected during the same cardiac cycle. In some embodiments, the first plurality and second plurality of subsets of self-gating data are collected during different cardiac cycles.

In some embodiments, the method further comprises a step of acquiring a training dataset comprising one or more subsets of training data, prior to the acquisition of the plurality of subsets of self-gating data.

In some embodiments, the training dataset is collected from a single cardiac cycle or a plurality of consecutive cardiac cycles. In some embodiments, the training dataset is collected from a plurality of non-consecutive cardiac cycles.

In some embodiments, the training dataset is processed based on one or more training algorithms to produce a training result.

In some embodiments, the one or more training algorithms comprises principal component analysis, multilinear principal component analysis, a machine learning technique, independent component analysis (ICA), clustering analysis, analysis of variance (ANOVA) analysis, blind deconvolution, factor analysis, multilinear subspace learning, non-negative matrix factorization (NMF), nonlinear dimensionality reduction analysis, projection pursuit analysis, Varimax rotation analysis, and a combination thereof.

In some embodiments, the training result is selected from the group consisting of a principal component vector, a threshold for detecting a triggering event, an expected duration of a cardiac cycle, a parameter associated with an imaging device that is used for collecting the training dataset, and combinations thereof.

In some embodiments, the method further comprises a step of processing the one or more subsets of training data, based on one or more training algorithms.

In some embodiments, the plurality of subsets of self-gating data is processed based on the training result to detect the presence of a triggering event.

In some embodiments, the method further comprises a step of processing the plurality of subsets of self-gating data, based on the training result to detect the presence of the triggering event.

In some embodiments, the method further comprises a step of initiating image acquisition, upon detection of the onset of the triggering event.

In some embodiments, the triggering event is the onset of a heartbeat.

Also provided herein is a data collection sequence for Magnetic Resonance Imaging (MRI) data acquisition. The data collection sequence comprises: a plurality of collection cycles, wherein at least one collection cycle in the plurality of collection cycles comprises: a self-gating mode during which self-gating data is collected; and an imaging mode during which image data is collected. In some embodiments, the self-gating mode and the imaging mode in the at least one collection cycle do not overlap, and wherein non-phase encoded data of k-space center line is repeatedly acquired in the self-gating mode.

In some embodiments, the at least one collection cycle corresponds to a cardiac cycle. In some embodiments, the self-gating data is acquired using a plurality of radio frequency (RF) coil arrays. In some embodiments, the self-gating data is non-phase encoded.

In some embodiments, the training data is acquired using a plurality of radio frequency (RF) coil arrays. In some embodiments, the method further comprises a step of a training phase wherein training data is collected.

In some embodiments, the training phase covers the duration of one or more cardiac cycles.

It will be understood that any applicable embodiments described can be combined or used as alternatives, even with respect to different aspects of the invention.

### BRIEF DESCRIPTION OF THE DRAWINGS

Those of skill in the art will understand that the drawings, described below, are for illustrative purposes only. The drawings are not intended to limit the scope of the present teachings in any way.

FIG. 1 illustrates an exemplary diagram of the proposed cardiac self-gating pulse sequence. RF: radio frequency; PE: phase encoded; and RO: readout.

FIG. 2 illustrates A) exemplary process/algorithm for self-gating signal processing and image reconstruction; and B) an exemplary computer system for implementation.

FIG. 3 illustrates exemplary cardiac self-gating signals derived using k-space center from a slice in short-axis view: a) imaging phase-encoding gradients turned on; b) imaging phase-coding gradients turned off to eliminate eddy current effects.

FIG. 4 illustrates representative cardiac gating signals. Cardiac gating signals derived from k-space center (top row) and the proposed MOCCA method (2^{nd} row) acquired in a four-chamber slice orientation. MOCCA signal is clearly better with all trigger position accurately detected compared to ECG signal reference (3^{rd} row). Center of k-space signal is not able to provide accurate trigger signal. *: triggers from MOCCA

FIG. 5 illustrates an exemplary cardiac self-gating sequence. Non-phase-encoded k-space center lines (non-PE line) are continuously acquired following cine-type or non-cine imaging acquisition. The MOCCA self-gating technique is applied on the non-PE lines until the new trigger is detected, at which time the self-gating is terminated and the imaging acquisition for the next k-space segment is initiated.

FIG. 6 illustrates an exemplary MOCCA self-gating algorithm. The L-2 norm of complex differences between MOCCA echoes and MOCCA echo reference is used as self-gating signal. The MOCCA echo reference is updated upon detection of new self-gating trigger signal.

FIG. 7 illustrates modified cardiac CINE sequence with multiple dedicated self-gating acquisitions (k-space center line with PE off) added at the end of each imaging window. For validating the proposed method, the sequence is prospectively triggered by every other ECG triggers.

FIG. 8 illustrates results of exemplary analysis: (a) self-gating signal using k-space center point from radial acquisition 1 shows significant signal drifting and distortion even after a band-pass filter; and (b) self-gating signal using the proposed method (e.g., sequence shown in FIG. 7) without any frequency filtering is capable of offer accurate and stable cardiac triggers compared with ECG triggers.

FIG. 9 illustrates results of exemplary analysis: a) k-space center point from radial acquisition, which was used in conventional cardiac self-gating method as previously described, shows cardiac motion signal with severe drifting and distortion; b) self-gating signal and triggers (marked by “*”) detected by the proposed method on the same subject as in a); c) self-gating and d) ECG signal with triggers on a 3 T scanner where the ECG fails to provide accurate triggers while the proposed method offers stable triggers.

FIG. 10 illustrates results of exemplary analysis: a) k-space center point from radial acquisition, which was used in conventional cardiac self-gating method), shows cardiac motion signal with severe drifting and distortion; b) self-gating signal and triggers (marked by “*”) detected by the proposed method on the same subject as in a). The detected self-gating trigger perfectly matches the corresponding ECG R wave (marked by ▾).

FIG. 11 shows Cardiac CINE images acquired by proposed self-gating sequence and standard ECG-gated sequence (4 out of 17 cardiac phase s are selected for display).

FIG. 12 illustrates an exemplary embodiment, showing K-space center point and corresponding ECG signals from (a) stationary phantom in a radial CINE sequence; (b) stationary phantom using a non-phase-encoded Cartesian CINE sequence; (c) in-vivo using a radial CINE sequence and (d) in-vivo using a non-phase-encoded Cartesian CINE sequence. Center point signal in (a) and (c) shows distortion as addressed in the hypothesis. Signal in (b) and (d) is free of the aforementioned distortion although mixed with noise.

FIG. 13 illustrates an exemplary embodiment, using MOCCA echo as the self-gating data where a MOCCA echo is formed by concatenating k-space centerline from different coils into a single column vector \vec{S}.

FIG. 14 illustrates an exemplary embodiment, showing a step-by-step illustration of PCA algorithm used for self-gating data processing. The training phase has 3 steps: the formation of a training matrix (1), the calculation of its covariance matrix (2) and the Eigen-decomposition (3) to derive the first Eigen-vector q1. The projection phase is a simple linear projection of new MOCCA echoes vector onto the first Eigen-vector q1 using vector dot product.

FIG. 15 illustrates an exemplary embodiment, showing an implementation of the proposed sequence. The scanner sends the measurement data of each line to the Image Reconstruction System where the self-gating data processing is performed. Once a trigger is detected, the image reconstruction system sends a real-time feedback to the scanner control computer, which switch to imaging mode.

FIG. 16 illustrates an exemplary embodiment, showing selected principal component (PC1, 2, 3, 5, 10) of the self-gating data and their contribution to overall signal variance. Note that the plots have different scales in y-axis. The first principal component is chosen because it best measures the cardiac motion and contributes more than 60% of the total signal variance. Other principal components show different level of noise.

FIG. 17 illustrates an exemplary embodiment, showing MOCCA self-gating signal after PCA processing with the triggers marked using triangle and the corresponding ECG signal and triggers recorded during scan from (a) 1.5 Tesla scanner using short axis view. (b) 3 Tesla scanner using vertical long axis view.

FIG. 18 illustrates an exemplary embodiment, showing selected cine images in short axis view from systole to diastole acquired using conventional ECG-gated bSSFP sequence (a-d) and self-gated bSSFP sequence (e-h) on the same subject using a 1.5 T scanner. (i) Plot of Recorded ECG signal and scan mode switching of self-gated sequence based on the time stamps recorded for these signals. No major difference in terms of image quality can be observed between self-gated and ECG-gated images. The scan mode switching was synchronized with the ECG R-wave although there is a noticeable delay between self-gating triggers and ECG triggers.

FIG. 19 illustrates an exemplary embodiment, showing selected cine images in vertical long axis view from systole to diastole acquired using conventional ECG-gated bSSFP sequence (a-d) and self-gated bSSFP sequence (e-h) on the same subject using a 1.5 T scanner. (i) Plot of recorded ECG signal and scan mode switching of self-gated sequence based on the time stamps recorded for these signals. No major difference in terms of image quality can be observed between self-gated and ECG-gated images. The scan mode switching was synchronized with the ECG R-wave although there is a noticeable delay between self-gating triggers and ECG triggers.

### DETAILED DESCRIPTION

### Self-Gating

Cardiac MRI scan methods without ECG signals are known in the art. Larson et al. proposed method of self-gated cardiac cine MRI in which the k-space center point from radial acquisition is used as the self-gating signal to measure the cardiac motion. Additional studies proposed a different strategy by using the k-space center line instead of k-space center point as the self-gating signal. The work represents the state of the art for this research area. More details can be found in Larson A C et al., 2004, “Self-gated cardiac cine MRI,” Magn Reson Med 51(1):93-102; Crowe M E et al., 2004, “Automated rectilinear self-gated cardiac cine imaging,” Magn Reson Med 52(4):782-788; and Nijm G M et al., 2008, “Comparison of self-gated cine MRI retrospective cardiac synchronization algorithms,” Journal of Magnetic Resonance Imaging 28(3): 767-772, each of which is hereby incorporated by reference in its entirety.

These known methods, however, either suffer from extended acquisition time or are limited to radial acquisition and often affected by eddy-current induced artifacts. In addition, the methods used retrospectively gated sequence that requires copying data to a separate computer for post-processing in order to get the image.

Provided herein are methods for proving cardiac synchronization for imaging process without ECG signals. Instead of using ECG, signals acquired by the RF (radio frequency) coil arrays are used to provide cardiac synchronization for the imaging process. This is achieved by adding, to a standard cardiac MRI pulse sequence, a special designed “Self-Gating Mode,” where non-phase encoded k-space center line is repeatedly acquired. The signal acquired in the “Self-Gating Mode” is processed by machine learning algorithms to estimate the cardiac motion and control the timing of the imaging pulse sequence.

Advantageously, the presented invention can be an alternative or replacement of ECG in almost all clinical cardiac MRI applications (e.g., cardiac CINE), in which the required set-up time of each individual patient is greatly reduced, leading to a more efficient and less expensive cardiac MRI scan. Another promising direction towards the application of this invention is the up-coming high magnetic field MRI (7 Tesla and up) where ECG devices often fail to provide stable and accurate cardiac synchronization signal due to interference with the high field.

Also advantageously, the invention could be potentially applied in cardiac MRI for special individuals where a reliable ECG signal of the subject is not available. One of the most promising examples is fetal cardiac MRI. Currently, a high quality time-resolved fetal cardiac imaging is clinically unavailable, mostly because the ECG of the fetus is inaccessible. The presented self-gating technique in this invention provides the otherwise unavailable real-time fetal cardiac motion measurement, making it possible to acquire high-quality fetal cardiac imaging, which is of significant clinical value.

Also advantageously, the application of the presented invention is not limited to cardiac MRI. The same scheme and technique with some minor variation could be applied to other motion sensitive MRI applications. (e.g., respiratory self-gated MRI, respiratory and cardiac dual self-gated MRI, patient body motion correction, etc.)

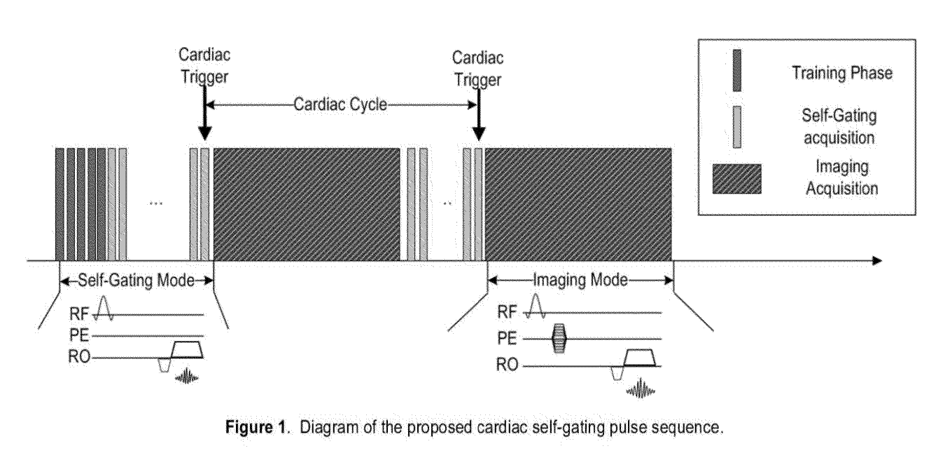

In one aspect, the cardiac self-gating method disclosed herein introduces a “self-gating mode” into a standard cardiac MRI pulse sequence (e.g., FIG. 1). The self-gating acquisitions are separated from imaging acquisitions and the difference between the two in terms of RF pulse and magnetic gradients are kept to a minimum. This is to avoid interference between the two modes, which otherwise could result in inaccurate cardiac gating or reduced image quality.

In some embodiments, the method provided herein is a combination of a modified cardiac MRI pulse sequence running on the scanner and a real-time signal processing software running on the online image reconstruction computer.

A conventional MRI system consists of two parts: 1) a scanning device and its controller and a computing device for image reconstruction. In some embodiments, the scanning device is a scanner that includes RF transmission coils, receiving coils, main magnetic field, magnetic field gradient etc. In some embodiments, the pulse sequence (FIG. 1) is installed on the scanner to control the different components to acquire MRI signals. In some embodiments, the computing device is an online image reconstruction computer. The computer receives MRI signals acquired by the scanner and performs image reconstruction and calculation. The output from the computing device is an MRI image.

In some embodiments, the pulse sequence comprises a non-phase encoded self-gating mode and an imaging mode. The structure of an exemplary pulse sequence is depicted in FIG. 1. A modified cardiac CINE sequence with added “self-gating mode” where k-space center line is repeated acquired. The sequence switches from “self-gating mode” to “imaging mode” once a new cardiac trigger is detected and switches back after the current imaging acquisition is finished.

In some embodiments, self-gating and imaging acquisition differs in that the self-gating acquisition is without the phase encoding gradient. ReadOut (RO) gradient and RadioFrequency (RF) pulse are kept the same (e.g., FIG. 1). In MRI pulse sequence, each component (e.g., RF) in the current acquisition method can cause some unwanted interference to the following few acquisitions unless all the components are kept the same so that a “steady-state” is reached.

If a different RF is used with different parameters (e.g., EchoTime: TE; RepetitionTime: TR) and different gradients (ReadOut:RO or Phase Encoded:PE) for the self-gating acquisition, the steady-state is broke and interference between the self-gating acquisition and imaging acquisition can cause inaccurate cardiac synchronization and compromised image quality. Thus, in the current self-gating method, the difference between the self-gating mode and imaging mode is kept at a minimum. The PE gradient does not cause much interference. As such, the following are achieved: 1) self-gating signals free of any distortion and artifact and 2) image of quality that are equivalent or superior to ECG-gated images.

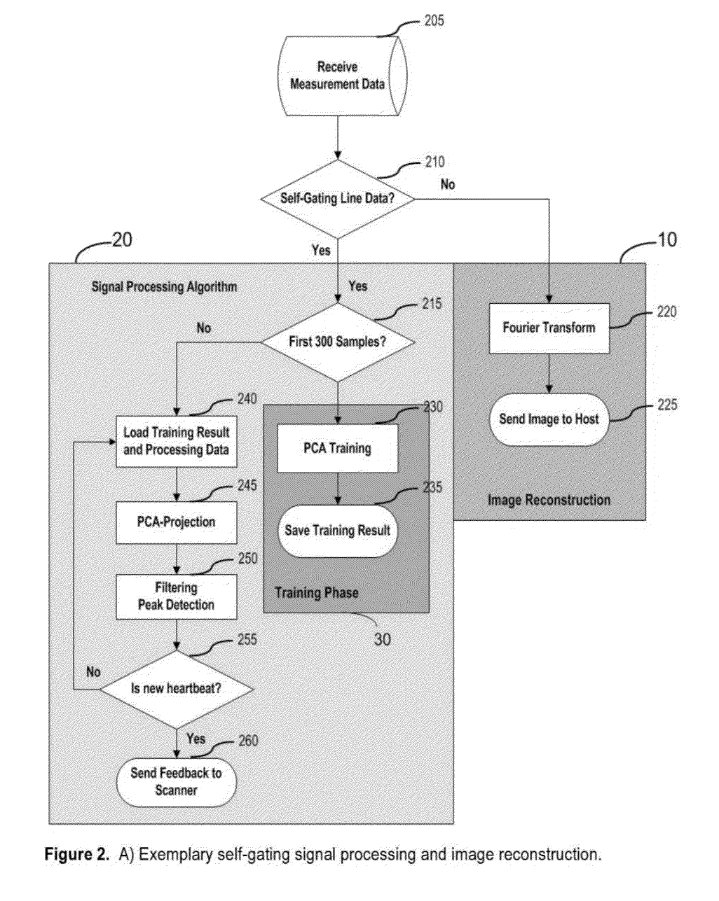

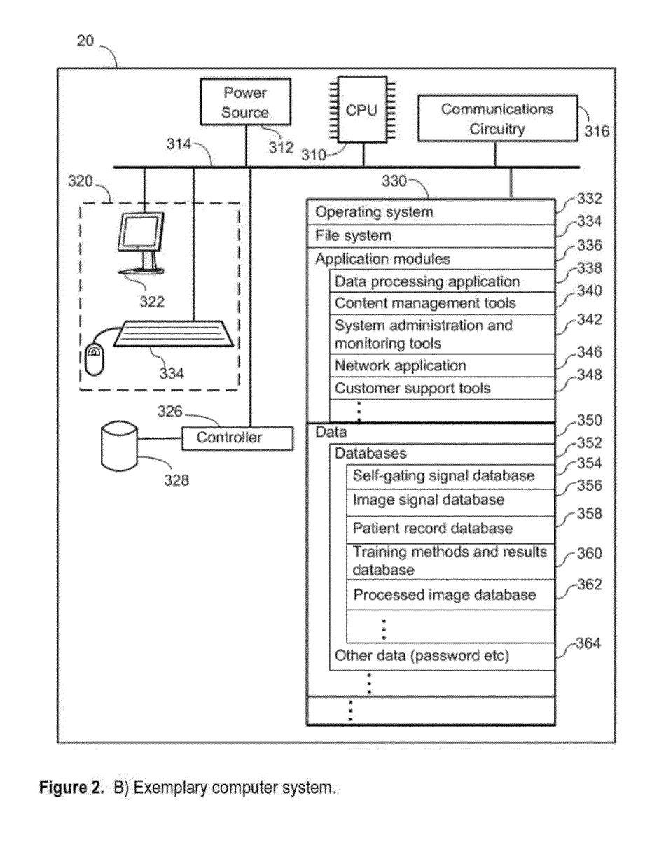

In some embodiments, the sequence switches between a non-phase encoded self-gating mode and an imaging mode where the segmented Cartesian K-space acquisition is performed. In some embodiments, an imaging mode is triggered when a cardiac trigger (e.g., the onset of a new heartbeat) is identified; for example, using the signal processing algorithm shown in FIG. 2.

In some embodiments, the sequence switches back to self-gating mode once the image acquisition is done. In some embodiments, an image acquisition window is set during the training phase which is shorter than the expected cardiac cycle. For example, a cardiac cycle is 1000 ms, the image acquisition window can be set to 900 ms. In some embodiments, imaging acquisition is initiated when a heartbeat is detected by self-gating and ended before the next heartbeat. The sequence switches back to self-gating mode before the next heartbeat so that the next heartbeat can still be detected by the self-gating mode.

During the self-gating mode, the sequence runs under real-time schema and send the acquired data to the signal processing software. Whenever a cardiac synchronization signal is initiated by the signal processing software and received by the MRI scanner, the sequence immediately switches to the imaging mode. The duration of the imaging mode is set as approximately 85% of a cardiac cycle so that the sequence can switch back to self-gating mode before the next heartbeat.

In some embodiments, the potential information provided by multiple coil arrays is used to render a reliable cardiac motion estimation that is available in real-time.

In some embodiments, coil arrays are a standard component in conventional MRI systems that can be used in accordance with the present methods. In conventional MRI systems, signal is most commonly acquired by one or several RF coil arrays receivers. Multiple coils (coil arrays) are placed with different orientations as close as possible to the imaged organs to provide maximum signal-to-noise ratio (SNR).

Previous self-gating methods discard specific information from data acquired by coil arrays according to one of the two patterns: only one coil is chosen and the data from other coils are discarded simply add the data from all coils together and assume it as one coil.

In some embodiments, the MOCCA technique is used to rearrange the data acquired by coil arrays. In some embodiments, a self-gating signal processing algorithm (e.g., PCA, a machine learning technique) can make use of the information provided by coil arrays to provide more accurate cardiac motion measurement.

The exact placement of coil arrays is different in each individual patient. In some embodiments, a flexible algorithm (parameters) is used for processing the data acquired by coil arrays. For example, in a machine learning algorithm, the parameters can be automatically adjusted during the training phase so that the algorithm is individually tailored for each patient and each scan.

Provided herein are methods for optimizing the technical strategies for deriving accurate and reliable cardiac self-gating signals for imaging technologies (e.g., fetal cardiac MRI). Several approaches are investigated to refine the ability to derive cardiac self-gating signal in the context of fetal cardiac MRI, though one of skill in the art will understand that the approaches are applicable to all imaging technologies.

### Training Phase, Self-Gating Phase, and Imaging Phase

Provided herein are methods and data collection sequences that separate data collection into multiple phases. In some embodiments, a collection sequence comprises multiple cycles. In some embodiments, each of the cycles corresponds to the duration between two triggering events (e.g., a heartbeat). For example, in preferred embodiments, a data collection cycle corresponds to a cardiac cycle between two consecutive heartbeats. In some embodiments, a data collection sequence comprises one or more training phase where one or more training datasets are collected. In some embodiments, a data collection sequence comprises one or more self-gating phase where one or more self-gating datasets are collected. In some embodiments, a data collection sequence comprises one or more imaging phase where one or more imaging datasets are collected.

In some embodiments, a training phase is added, where a training dataset is collected and processed. A training dataset can be used to find an optimal way to represent the cardiac motion for each patient and each scan so that the parameters for the subsequent module (self-gating) can be individually tailored to maximize performance and reliability. Thus, preferably, a training dataset can be collected prior to collecting any actual dataset (e.g., self-gating or imaging). In some embodiments, a training dataset contains data collected from the same patient over one or more cardiac cycles; for example, 2 or more, 3 or more, 4 or more, 5 or more, 6 or more, 7 or more, 8 or more, 9 or more, 10 or more, 15 or more, 20 or more, 50 or more, and etc.

In some embodiments, training datasets from different patients can be used to extract machine specific information that is independent of patient characteristics. For such purposes, 2 or more, 3 or more, 4 or more, 5 or more, 6 or more, 7 or more, 8 or more, 9 or more, 10 or more, 15 or more, 20 or more, 50 or more datasets and etc. can be used.

In some embodiments, training datasets can be collected multiple times for iterative processing and optimization of parameters representing the cardiac motion of a patient and a scan. In some embodiments, multiple training datasets are collected over consecutive cardiac cycles. In some embodiments, multiple training datasets are collected over non-consecutive cardiac cycles. Exemplary parameters include but are not limited to a principle component vector, a threshold for trigger detection, an expected duration of a cardiac cycle, a parameter associated with an imaging device that is used for collecting the training dataset, and etc. In some embodiments, when multiple training datasets are collected, one or more average values can be computed for any or all of the parameters.

In some embodiments, a cardiac cycle is divided into a self-gating phase and an imaging phase. In some embodiments, a cardiac cycle is divided into one or more self-gating phases and one or more imaging phases. In some embodiments, a cardiac cycle is divided into one or more self-gating phases. In some embodiments, a cardiac cycle is one or more imaging phases. The number of self-gating or imaging phase can vary with respect to patients and/or equipment. For example, a cardiac cycle can be divided into 2 or more, 3 or more, 4 or more, 5 or more, 6 or more, 7 or more, 8 or more, 9 or more, 10 or more, 15 or more, 20 or more, 50 or more self-gating or imaging phases. A cardiac cycle can also be divided into 2 or more, 3 or more, 4 or more, 5 or more, 6 or more, 7 or more, 8 or more, 9 or more, 10 or more, 15 or more, 20 or more, 50 or more self-gating and imaging phases.

In some embodiments, a self-gating phase or an imaging phase can cover multiple cardiac cycles, for example 2 or more, 3 or more, 4 or more, 5 or more, 6 or more, 7 or more, 8 or more, 9 or more, 10 or more, 15 or more, 20 or more, 50 or more cardiac cycles.

In some embodiments, self-gating datasets can be collected multiple times for iterative processing and optimization of parameters representing the cardiac motion of a patient and a scan. In some embodiments, multiple self-gating datasets are collected over consecutive cardiac cycles. In some embodiments, multiple self-gating datasets are collected over non-consecutive cardiac cycles.

In some embodiments, imaging datasets can be collected multiple times based on the parameters extracted from the training and self-gating datasets. In some embodiments, multiple imaging datasets are collected over consecutive cardiac cycles. In some embodiments, multiple imaging datasets are collected over non-consecutive cardiac cycles.

In some embodiments, imaging datasets can be collected multiple times for iterative processing and optimization of parameters representing the cardiac motion of a patient and a scan. In some embodiments, multiple imaging datasets are collected over consecutive cardiac cycles. In some embodiments, multiple imaging datasets are collected over non-consecutive cardiac cycles. Exemplary parameters include but are not limited to a principle component vector, a threshold for trigger detection, an expected duration of a cardiac cycle, a parameter associated with an imaging device that is used for collecting the training dataset, and etc. In some embodiments, when multiple imaging datasets are collected, one or more average values can be computed for any or all of the parameters.

Any applicable algorithms can be used for processing the training dataset, self-gating dataset or imaging dataset. Exemplary processing algorithms include but are not limited to principal component analysis, multilinear principal component analysis, a machine learning technique, independent component analysis (ICA), clustering analysis, analysis of variance (ANOVA) analysis, blind deconvolution analysis, factor analysis, multilinear subspace learning analysis, non-negative matrix factorization (NMF) analysis, nonlinear dimensionality reduction analysis, projection pursuit analysis, Varimax rotation analysis, or a combination thereof.

### Data Separation

In one aspect, the method disclosed herein separates imaging data acquisition from self-gating data.

In some embodiments, the self-gating data acquisition is separated from the actual imaging data. In some embodiments, the self-gating data are acquired after the imaging data to eliminate self-gating signal distortions.

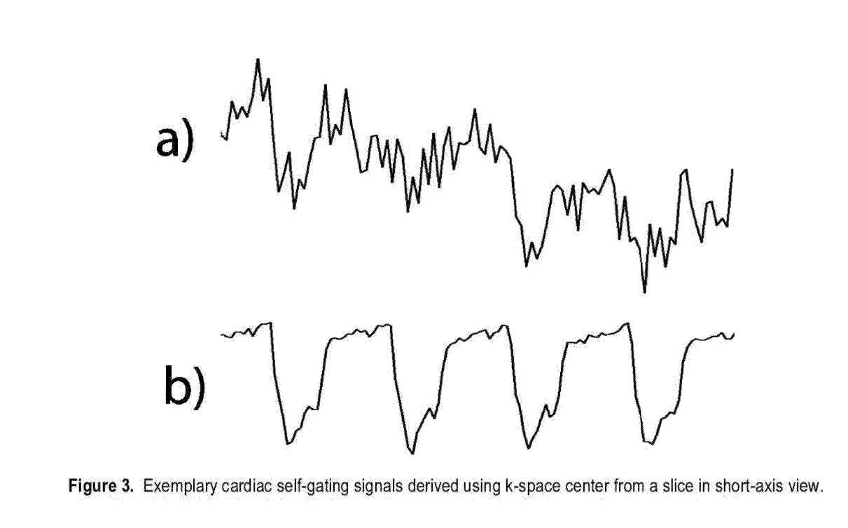

Existing cardiac self-gating methods for cine-type acquisitions acquire the self-gating data and imaging data within the same repetition time (TR) or successive TRs. Based on preliminary results, this design suffers from self-gating signal distortions that arise from varying eddy currents from changing phase-encoding (PE) gradients in imaging data acquisition (e.g., FIG. 3). As a result, the images are subject to cardiac motion artifacts due to inaccurate/unreliable trigger signals from the distorted self-gating signal. Furthermore, there has been no known cardiac self-gating method for non-cine acquisitions.

As noted, the quality of cardiac self-gating signal is heavily affected by eddy current effects (e.g., FIG. 3). During a normal imaging scan, PE gradients are varied to fill in the k-space lines. As a result, the self-gating signal acquired immediately after a PE line will be subject to a phase error caused by eddy currents that is different from the signal acquired after a different PE line. This effect tends to be more severe in steady-state free precession (SSFP) sequences due to the its fully balanced gradients (17).

Previously proposed techniques acquire the cardiac self-gating data either as part of the normal imaging data (9), during the same TR as the normal imaging data (9-11, 18), or acquired immediately after the normal imaging data (19). For example, Larson et al. (9) proposed a radial acquisition scheme, where the k-space center point is acquired in every radial k-space line during normal imaging data acquisition and these center points are subsequently used as a basis for deriving cardiac self-gating signal. In the method reported by Crowe (10) et al., the slice gradient is delayed to allow acquisition of the k-space center point as the self-gating data within the same TR as the normal imaging data. This strategy was also recently evaluated in fetal cardiac self-gating (15). Spraggins et al. (20) developed a technique where the self-gating data acquisition was interleaved with normal imaging data.

No prior studies investigated the aforementioned eddy current effects on self-gating signals. Current data indicate this effect is potentially significant. In cardiac self-gating applications, where reliability and robustness are dominant factors that determines its clinical utility, such a source of self-gating signal distortion and drifting needs to be addressed.

In some embodiments, the method disclosed herein proposes to continuously acquire the self-gating data after the normal imaging data until the next trigger signal is detected. It is hypothesized that the proposed design will eliminate the undesirable eddy current effect. Additionally, all previous cardiac self-gating methods are designed for cine-type acquisitions, and are hence not readily available for non-cine acquisitions. The method can be easily applied in all cine (or time-resolved within the cardiac cycle) and non-cine cardiac imaging acquisitions.

Exemplary Process for Separating Imaging Data Acquisition from Self-Gating Data

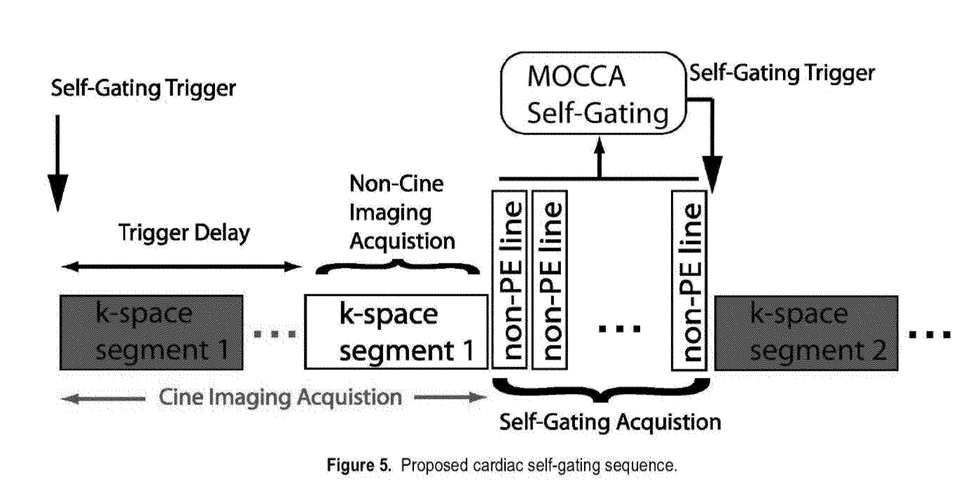

FIG. 1 shows that the cardiac self-gating signal is degraded by eddy current effects of varying PE gradient amplitudes from TR to TR. A high quality self-gating signal was generated from an acquisition where the PE gradients were turned off. A strategy was proposed such that non-phase-encoded signals are acquired continuously, based on which the self-gating signal is derived. This acquisition starts immediately after the end of imaging data acquisition and is terminated upon detection of the new trigger, as shown in FIG. 5. Such a design, where the imaging data and self-gating data acquisitions are separate in time within the cardiac cycle, eliminates the aforementioned signal degradations and is readily available for both cine and non-cine acquisitions.

In some embodiments, the approach outlined above will be tested on healthy adult volunteers (e.g., 20 or more; 30 or more; 40 or more; 50 or more; 60 or more; 80 or more; 100 or more). On each volunteer, the ECG signal will be used to provide triggers, but retrospectively evaluate the trigger position from self-gating methods.

In some embodiments, the approach outlined above will be tested on fetuses (e.g., 20 or more; 30 or more; 40 or more; 50 or more; 60 or more; 80 or more; 100 or more).

The following types sequences will be tested on these volunteers: 1) The cardiac cine MRI sequence used in the preliminary studies where imaging data and self-gating data are acquired in an interleaved fashion; 2) A ECG-triggered 2D black-blood turbo spin echo (TSE) sequence with the proposed method; 3) A retrospectively ECG-gated cardiac cine MRI sequence with the proposed method, where the k-space center line is acquired for self-gating ˜60 ms before the next expected ECG R wave. The subject's heart rate immediately before the scan will be used to calculate the time for the “next expected R wave.”

It is understood that the heart rate of a fetus (˜120 bpm) is much faster than a healthy adult subject; however, it should be straightforward to adapt the timing of the sequence to this issue. Each of the three sequences will be repeated 4 times to test reproducibility. In some embodiments, the raw data of all acquisitions will be exported into Matlab (MathWorks, Natick, Mass.), where cardiac self-gating signals will be retrospectively derived from each data set as follows.

Derivation of Cardiac Self-Gating Signal

The MOCCA algorithm (i.e. L-2 norm of complex differences between MOCCA echoes) will be used to derive the self-gating signals. The optimal use of multi-coil information will be further studied separately in subsequent sub-aim. In some embodiments (e.g., as shown in the preliminary data), no filtering is needed before the trigger position can be identified using the proposed acquisition approach, although filtering will be applied if needed. In some embodiments, one or more filtering mechanisms are applied.

To validate the accuracy of trigger position, the time differences between the ECG R wave and the triggers from the self-gating signals acquired using all three sequences will be analyzed using a repeated measures analysis of variance test. The sequences outlined in method 2 and 3 may be more accurate than method 1 because the effects of varying PE gradients are eliminated. The reliability and reproducibility of each method will also be assessed using the four repeated acquisitions. With a sample size of 20, preliminary data indicate an effect size of 0.73 can yield a power of 81% with a 5% level of significance.

For a cardiac-phase resolved acquisition, such as cine cardiac MRI or phase contrast flow imaging, such a design may possibly miss the last end-diastolic cardiac phase of the movie. This will unlikely be as a major issue and can be resolved by delaying the start of self-gating data acquisition to ensure coverage of the whole heart cycle, albeit at the cost of reduced scan time efficiency due to the need for spending a longer time waiting for the next trigger signal. Given the high heart rate of fetuses (˜120 bpm), acquisition can be accomplished within a single maternal breath-hold.

In an SSFP sequence, paired phase-encodes (17) has been proposed to reduce the effect of eddy current phase errors, which can serve as an alternative approach if the proposed method is not adequate.

Full k-Space Center Lines and Multi-Coil Arrays

Provided herein are self-gating methods that include data from the full k-space center line rather than a single k-space center point. Previously proposed cardiac self-gating methods use the single k-space center point as the self-gating signals. It has been demonstrated that including the full k-space center line rather than a single k-space center point results in more reliable self-gating.

Also provided herein is a MOtion Compensation technique with Coil Arrays (MOCCA) for self-gating; e.g., cardiac or respiratory self-gating, where the coils are used as multiple motion “sensors” to take advantage of the additional information offered by the localized coil sensitivity profiles.

In some embodiments data from full k-space center lines are used to generate the self-gating signals. In some embodiments data from multi-coil signals are used to generate the self-gating signals. In some embodiments data from full k-space center lines and multi-coil signals are used to generate the self-gating signals.

All previously proposed cardiac self-gating methods uses the k-space center point only; however, it has been demonstrated in the preliminary study that inclusion of the full k-space center line will greatly reduce fluctuations in the self-gating signal. With the advances of modern MRI systems with multi-receiver capabilities, most cardiac MRI exams are now performed using multi-coil arrays. Due to the localized coil sensitivities, the motion-induced signal variations from the receiver coils are subject to modulations from their individual coil sensitivities. Although the localized coil sensitivities have been extensively used in parallel imaging to shorten imaging time, their benefits in motion correction, especially in self-gating, have not been well studied. The recently proposed cardiac self-gating approaches are based on signal from a single chosen coil within the array (usually the signal with the maximum signal amplitude).

It is hypothesized that the localized coil sensitivities helps to better detect and gate the motion by improving the reliability for the self-gating signal.

In some embodiments, techniques for motion correction using coil arrays are applied (e.g., MOCCA). In MOCCA, the coil arrays are used as multiple “sensors” of motion and the coil-dependent motion-induced signal variations are used to achieve the above benefits. For example, MOCCA technique was shown to be valuable for respiratory self-gated free-breathing cardiac cine MRI applications (21).

In some embodiments, two or more coil arrays are used. In some embodiments, the coil arrays includes three or more, four or more, five or more, six or more, seven or more, eight or more, nine or more, 10 or more, 12 or more, 15 or more, 16 or more, 18 or more, 20 or more, 24 or more, 28 or more, 30 or more, 35 or more, 40 or more, 50 or more, 60 or more, 80 or more, 100 or more coil arrays.

In some embodiments, MOCCA techniques are used in fetal cardiac self-gating. In some embodiments, MOCCA techniques are used to detect bulk fetal motion during imaging. In some embodiments, MOCCA techniques are used for motion compensation. In some embodiments, MOCCA techniques are used for both self-gating and motion compensation

### Optimization for Improved Gating Signal Quality.

Prior investigators have developed cardiac self-gating methods using the single k-space center peak point only (9-11). In MOCCA, the non-phase-encoded k-space center line (MOCCA line) is used instead of the k-space center point. Furthermore, multiple coils are included instead of a single coil as previously proposed. The inclusion of multiple coils and a full k-space center line is advantageous and will improve fetal cardiac self-gating signal. This point is demonstrated in the preliminary results of FIGS. 3 & 4.

Here, the work on MOCCA respiratory self-gating will be extended (21). In addition to “stacking up” the k-space center lines from multiple coils into a MOCCA echo, as used in the preliminary study, the effects of a weighted average of self-gating signals from all the receiver coils will be analyzed.

In some embodiments, phase information will also be included in derivation of cardiac self-gating signal. Previous cardiac self-gating methods used the magnitude of the k-space center point. As an object moves relative to the coils, as is the case in cardiac motion, the motion causes changes not only in signal magnitude but also in phase. The phase change has two components: 1) the phase variation governed by the k-space linear phase ramp caused by a translation in image space, and 2) the phase change caused by the relative motion between the object and the spatial profile of coil sensitivity. These changes in signal phase are less appreciated when only the k-space center point is used. Therefore, by including the whole k-space center line, the phase changes caused by cardiac motion can be better utilized.

Another benefit of using full k-space center lines instead of k-space center point only is that it allows us to focus better on the fetal heart region. Compared to cardiac self-gating for adults, fetal cardiac self-gating may be more sensitive to interference from the much stronger maternal signal. Thus, in some embodiments, self-gating signals are based on data more localized to the fetal heart. In the slice encoding direction, the self-gating data should be confined only to the relevant fetal heart anatomy. To further localize in the readout direction, the FOV in the readout direction is reduced to the size of the fetal heart and potentially the fetal lungs, which are filled with amniotic fluid is bright in MRI, by filtering the non-PE lines before further processing.

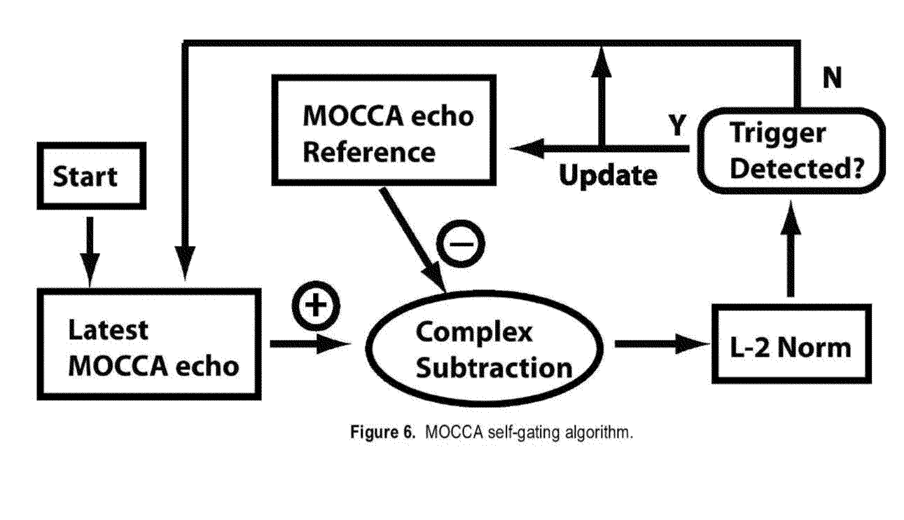

As shown in FIG. 5, the self-gating data is acquired separately in time to the imaging part. Here, the cardiac trigger signal will be provided by ECG, and the self-gating signal will only be retrospectively calculated and compared with ECG trigger positions. The cardiac self-gating signal will be generated using the method described in Preliminary Studies and depicted in FIG. 6. Furthermore, the self-gating signals separately will be generated for each coil and linearly combine the resultant self-gating signal using the maximum signal amplitude of the no-PE line as the coil weights. As a comparison, another self-gating signal will be derived as the magnitude of the k-space center point only, as proposed by several previous investigators (9). The two flavors of MOCCA methods will also be re-applied on the filtered k-space center lines corresponding to the fetal heart/lung region. Each of the six self-gating signals will then undergo a peak detection algorithm used by Larson et al. (9).

Experiments will be performed on 20 healthy adult volunteers. Both cine-type and non-cine acquisitions will be performed. For the cine MRI acquisition, each subject will be imaged three times in the standard short-axis, two-chamber and four-chamber views. The non-cine TSE sequence will be performed in the four-chamber view. The temporal fidelity of the three self-gating methods (MOCCA based on “stacking up” non-PE lines, MOCCA based on weighted linear combinations, and previous k-space center point only approach) will be compared to the ECG trigger positions using repeated measures analysis of variance test. With a sample size of 20, preliminary data indicate an effect size of 0.73 would yield a power of 81% with a 5% level of significance.

The combination of multiple coils with a full non-PE line leads to better temporal fidelity in the self-gating trigger positions compared with k-space center point only approach. The superiority can be tested using the McNemar's test. Based on statistics, it is possible to choose one of the two MOCCA self-gating methods for subsequent analysis.

Eliminating maternal signal interference in the readout direction can also lead to better accuracy for trigger detection. Furthermore, the time stamps of the detected triggers within the cardiac cycle will be examined and the relation between the morphology of the self-gating signal to the slice orientation will be analyzed. Previously proposed cardiac self-gating methods lead to highly variable morphology that is heavily dependent on subject and slice orientations (9). These previous methods tend to work better in the short axis orientation due to the more significant change in blood volume (which has high signal) in that orientation. The preliminary data demonstrated that the morphology self-gating signal generated by the proposed methods is independent from the imaging slice orientation and the detected peaks correspond precisely to the ECG R wave in the two common slice orientations tested. These properties will greatly facilitate reliable automatic peak detection algorithms and their practical implementations on clinical scanners.

The proposed cardiac self-gating method does not address bulk motion of the fetus, which is another potential source of artifacts and image blurring. As another benefit of using multiple coils, bulk fetal body motion may be better detected by examining the self-gating signals. By using multiple “sensors”, the motion of the fetal head and extremities may be better detected by the MOCCA echo. Therefore, the MOCCA echoes will be examined and detect MOCCA echoes that have a cross correlation (or Euclidean distance) that is out of range of the previous heart cycle, in which case the sequence will be repeated until no fetal bulk motion is detected by the MOCCA echoes in the new acquisition.

### Data Localization

In some embodiments, self-gating signals are localized to the region of the fetal heart. The rationale is that the self-gating signal from the fetal heart is significantly smaller than the maternal signal due to its small size. Even with a maternal breath-hold, motion of the mother's abdominal organs other than respiratory motion will therefore interfere with the fetal cardiac self-gating signal. Therefore, it is possible to reduce these interferences from maternal signal by only “listening to” the signal from the fetal heart/lung region. The spatial localization in the slice-encoding direction will be achieved by the conventional slab/slice selection gradient. To achieve spatial localization in the frequency-encoding direction, data from the non-phase-encoded k-space center line will be acquired with the corresponding Field of View (FOV) set to the location of the fetal heart, since the FOV in the frequency-encoding direction can always be set to a small size with no aliasing artifacts. Localization to the fetal heart/lung region is only possible because full k-space center line data are used rather than a single k-space center point as previously proposed for cardiac self-gating. Spatial localization of the self-gating signal has not been previously studied and it is expected to be especially useful for fetal cardiac self-gating.

### Retrospective and Prospective Self-Gating

In one aspect, the method disclosed herein is based on a prospectively gated sequence, which offers a better image efficiency and gating accuracy over retrospectively gated sequence.

In some embodiments, the techniques described herein will be evaluated in a retrospective fashion to allow validation against the gold-standard ECG signal in healthy subjects. In some embodiments and to allow for clinical validation on fetuses, sequences that allow prospective cardiac self-gating on the fly will be developed with the proposed strategies and evaluate the prospectively self-gated cardiac images on healthy adult subjects.

Incorporating results from the retrospective self-gating techniques, a sequence module that prospectively uses cardiac self-gating will be developed for trigger detection in real time. The cardiac self-gating module will be integrated into 2D breath-held cine cardiac MRI and TSE sequences. The developed prospective cardiac self-gated sequences will then be tested on 20 healthy adult subjects. The goal here is to evaluate cardiac images acquired with self-gating and compare that with the ECG-gated images.

Each subject will be imaged using the cardiac self-gated cine MRI and TSE sequences in the short-axis and four-chamber views. Immediately following the self-gated acquisitions, the corresponding ECG-triggered sequences will then be performed on each subject. The order of acquisitions will be randomized.

The image quality assessments (on a 4 point scale) and quantitative blood-myocardial border sharpness measurements will be performed as previously proposed (9, 21) and the results will be compared using a paired t-test for sharpness scores and Wilcoxon signed rank test for image quality. The sample size of 20 gives a preliminary effect size of 0.75 will yield a power of 88% at the 5% significance level. The hypotheses to be tested are that the images will not be inferior to the conventional ECG-gated images. The non-inferiority will be tested using the Nam method (22).

### Data Processing

In one aspect, machine learning technique is implemented to process the self-gating signals (e.g., FIG. 2). Advantageously and in some embodiments, the learning-based algorithm can be used to individually tailor the processing algorithm based on each patient and each image orientation without user intervention.

A flowchart of an exemplary self-gating data processing software is shown in FIG. 2.

When a new measurement data is received, the software first determines whether it is a self-gating acquisition. If so, the data is fed to the self-gating algorithm path; otherwise, it is fed to the normal image reconstruction path. The self-gating algorithm takes the first 300 self-gating acquisitions as the training data for the machine learning based algorithm (PCA). Other statistical data such as expected cardiac cycle, peak detection threshold are also derived from the training data. Starting from the 301st self-gating acquisition, the algorithm uses the learned pattern from the training phase to process the signal. When a new heartbeat is detected, the algorithm sends a feedback signal to the scanner to trigger the pulse sequence. More details of the invention are described in the attached conference abstracts attached.

After measurement data are received at step 205, they are processed to determine whether they are self-gating line data or imaging date (e.g., step 210). Imaging data are transferred to an image reconstruction module 10. Algorithms such as Fourier Transformation are applied for image processing at step 220. The resulting image is sent to a host at step 225 or stored locally before further processing and/or optimization is applied.

Self-gating line data are transferred to a signal processing module 30 which comprises algorithms for both a training phase and an actual processing phase. In some embodiments, initial data (e.g., first 300 samples) are used to train the algorithm (e.g., step 215). For example, mathematical procedures (e.g., principal component analysis) are applied in the training algorithm (e.g., step 230). PCA uses an orthogonal transformation to convert a set of observations of possibly correlated variables into a set of values of linearly uncorrelated variables called principal components. Any applicable procedures or analyses can be used, including but not limited to grid analysis, gradient analysis, linear map analysis, transformation matrix analysis, multi-linear PCA, correspondence analysis, Eigenface analysis, exploratory factor analysis, geometric data analysis, factorial code, independent component analysis, Kernel PCA, Matrix decomposition, nonlinear dimensionality reduction, Point distribution model analysis, regression analysis, singular spectrum analysis, singular value decomposition, sparse PCA, transform coding, weighted or un-weighted least squares analysis, dynamic mode matrix factorization analysis. Training results are saved locally or on a host via network connection.

The subsequently collected data are further processed at step 240 based on results from the training analysis. Processing includes for example, PCA projection analysis at step 245 and filtering peak detection analysis at step 250. If a new heartbeat is detected at step 255, the feedback is send to an imaging device such as a scanner at step 260. The imaging device can be initiated and start imaging data acquisition. Alternatively, when a new heartbeat is not detected, the algorithm loops back to data processing at step 240. In some embodiments, additional data are used before further processing for heart beat detection. In some embodiments, no additional data is used; however, new processing algorithm is applied for heart beat detection. In some embodiments, both additional data and new processing algorithm are used for heart beat detection.

### Computer Implementation

FIG. 2B illustrates an exemplary computer system 20 that supports the functionality described above and detailed in sections below.

In some embodiments, data server 300 may comprise a central processing unit 310, a power source 312, a user interface 320, communications circuitry 316, a bus 314, a controller 326, an optional non-volatile storage 328, and at least one memory 330. In some embodiments, the data server can be located on a local computer associated with the imaging device and data acquisition device. Alternatively, the data server can be located on a remote server and communicate with the imaging device and data acquisition device remotely via network. In some embodiments, the data server, imaging device and data acquisition device form an integrated system.

Memory 330 may comprise volatile and non-volatile storage units, for example random-access memory (RAM), read-only memory (ROM), flash memory and the like. In preferred embodiments, memory 330 comprises high-speed RAM for storing system control programs, data, and application programs, e.g., programs and data loaded from non-volatile storage 328. It will be appreciated that at any given time, all or a portion of any of the modules or data structures in memory 330 can, in fact, be stored in memory 328.

User interface 320 may comprise one or more input devices 324, e.g., keyboard, key pad, mouse, scroll wheel, touchscreen, virtual touchscreen and the like, and a display 322 or other output device. A network interface card or other communication circuitry 316 may provide for connection to any wired or wireless communications network, which may include the Internet and/or any other wide area network, and in particular embodiments comprises a telephone network such as a mobile telephone network. Internal bus 314 provides for interconnection of the aforementioned elements of data server 300.

In some embodiments, operation of data server 300 is controlled primarily by operating system 332, which is executed by central processing unit 310. Operating system 332 can be stored in system memory 330. In addition to operating system 332, a typical implementation system memory 330 may include a file system 334 for controlling access to the various files and data structures used by the present invention, one or more application modules 336, and one or more databases or data modules 350.

In some embodiments in accordance with the present invention, applications modules 336 may comprise one or more of the following modules described below and illustrated in FIG. 2B.

Data Processing Application 338.

In some embodiments in accordance with the present invention, a data processing application 338 receives and processes gating or imaging data. Gating or imaging data are delivered to a data storage system (locally or via network) from coil arrays. Algorithms depicted in FIG. 2A, disclosed herein or

Content Management Tools 340.

In some embodiments, content management tools 340 are used to organize different forms of databases 352 into multiple databases, e.g., a self-gating signal database 354, an image signal database 356, a patient record database 358, and a training method and result 360. In some embodiments in accordance with the present invention, content management tools 340 are used to search and compare data.

The databases stored on data server comprise any form of data storage system including, but not limited to, a flat file, a relational database (SQL), and an on-line analytical processing (OLAP) database (MDX and/or variants thereof). In some specific embodiments, the databases are hierarchical OLAP cubes. In some embodiments, the databases each have a star schema that is not stored as a cube but has dimension tables that define hierarchy. Still further, in some embodiments, the databases have hierarchy that is not explicitly broken out in the underlying database or database schema (e g, dimension tables are not hierarchically arranged). In some embodiments, the databases in fact are not hosted on data server 300 but are in fact accessed by data server through a secure network interface. In such embodiments, security measures such as encryption is taken to secure the sensitive information stored in such databases.

System Administration and Monitoring Tools 342:

In some embodiments in accordance with the present invention, system administration and monitoring tools 342 administer and monitor all applications and data files of data server 300. Because security sensitive data such as biometric keys are stored on data server 300, it is important that access those files that are strictly controlled and monitored. System administration and monitoring tools 342 determine which servers or devices have access to data server 300. In some embodiments, security administration and monitoring is achieved by restricting data download access from data server 300 such that the data are protected against malicious Internet traffic. In some embodiments, system administration and monitoring tools 342 use more than one security measure to protect the data stored on data server 300. In some embodiments, a random rotational security system may be applied to safeguard the data stored on data server 300.

In some embodiments in accordance with the present invention, system administration and monitoring tools 342 communicate with other application modules on data server 300. In some embodiments, before a user device 10 is registered with data server 300, initial access to data server 300 is granted by a backup access key 260 that has been assigned to user device 10 along with an IPv6 address. In some embodiments, backup access key 260 is recognized and monitored by system administration and monitoring tools 342.

Network Application 346:

In some embodiments, network applications 346 connect a data server 300 with intermediary gateway servers. Referring to FIG. 2B, a data server 300 is connected to multiple types of gateway servers (e.g., network service providers 40, wireless service provides 50, banks 60, online stores 70, hospitals 80, and stores 90). These gateway servers have different types of network modules. Therefore, it is possible for network applications 346 on a data server 300 to be adapted to different types of network interfaces, for example, router based computer network interface, switch based phone like network interface, and cell tower based cell phone wireless network interface, for example, an 802.11 network or a Bluetooth network. In some embodiments in accordance with the present invention, upon recognition, a network application 346 receives data from intermediary gateway servers before it transfers the data to other application modules such as data processing application 338, content management tools 340, and system administration and monitoring tools 342.

Customer Support Tools 348:

Customer support tools 348 assist users with information or questions regarding their accounts, technical support, billing, etc.

In some embodiments, each of the data structures stored on centralized data server 300 is a single data structure. In other embodiments, any or all such data structures may comprise a plurality of data structures (e.g., databases, files, and archives) that may or may not all be stored on centralized data server 300. The one or more data modules 350 may include any number of content files 352 organized into different databases (or other forms of data structures) by content management tools 340.

In addition to the above-identified modules, data 350 may be stored on server 300. Such data comprises database 352 and other data 364. Exemplary database 352 (self-gating signal database 354, image signal database 356, patient record database 358, training methods and results database 360 and processed image database 362) are described below.

Self-Gating Signal Database 354:

In some embodiments, self-gating signals are stored in a database, either in raw or process form. In some embodiments, self-gating signals collected from the same patient in different sessions are stored together.

Image Signal Database 356:

In some embodiments, image signals are stored in a database, either in raw or process form. In some embodiments, image signals collected from the same patient in different sessions are stored together.

Patient Record Database 358:

In some embodiments, patient records are stored in a database. In some embodiments, patient records are be linked to self-gating signals and/or image signal data from the same patients.

Training Methods and Results Database 360:

In some embodiments, training methods used to processed the initial self-gating signals (e.g., first 300 samples) are stored in a database. In some embodiments, results from the training session are also stored.

Processed Image Database 362:

In some embodiments, processed images are stored in a database. In some embodiments, patient records are be linked to processed images from the same patients.

In some embodiments, databases on data server 300 are distributed to multiple sub-servers. In some embodiments, a sub-server hosts identical databases as those found on data server 300. In some embodiments, a sub-server hosts only a portion of the databases found on data server 300. In some embodiments, global access to a data server 300 is possible for users and devices (for self-gate signal or image signal collection) regardless of their locations.

It is to be appreciated that databases, especially patient record database 358, on data server 300 is protected by restricting access to only authorized users. In some embodiments, data download from data server 300 is prohibited.

### Software and Computer Program Product

In one aspect, provided herein are one or more software or computer program products for controlling data acquisition and/or data processing.

### System Integration

In one aspect, the method disclosed herein is fully implemented on a commercial MRI system (e.g., SIEMES Avanto/Trio System) without the need of additional hardware. High-quality images are readily available right after the scan is finished.

In some embodiments, the method disclosed herein can be applied in most clinical breath-hold cardiac MRI applications. For example, a sequence containing both “self-gating mode” and “imaging mode” is installed on the commercial MRI scanner and a program that utilize the proposed self-gating signal processing algorithm is installed on the MRI image reconstruction system. In the “self-gating mode,” the scanner sends the acquired self-gating signals to a gating signal processing software program for processing. In some embodiments, the software program is installed in the MRI image reconstruction system. Whenever a new heart beat is detected, the self-gating program sends a signal back to the MRI scanner to initiate the “imaging-mode.”

The self-gating part is fully automated. That means from the user-end, the proposed invention is operated with no difference from conventional ECG-gated cardiac MRI sequence and is capable of providing cardiac MR images of similar quality immediately after the scans.

### Clinical Applications

Congenital heart disease (CHD) is the most common congenital defect affecting eight per thousand live births in North America (1). Prenatal diagnosis of CHD allows for more informed decisions on patient management before and after birth. In current clinical practices, an ultrasound examination of the anatomy and function of the heart as well as the blood flow through the valves, ductus arteriosus, and great vessels is usually used for prenatal diagnosis of CHD (2). However, the use of ultrasound is limited in certain patients due to maternal obesity (3), oligohydramnios (4), or issues with fetal position. Fetuses in their third trimester tend to be more difficult to assess using ultrasound compared to second trimester due to ossified bones and decreased amniotic fluid. Furthermore, assessment of certain diseases, e.g., fetal aortic coarctation, tends to be more difficult with ultrasound. The evaluation of fetal blood flow using Doppler requires assumptions about the shape of the vessel, angle of the transducer and the velocity profiles, all of which are potential sources of error in calculating flow. Fetal cardiac MRI is a promising imaging modality complementary to ultrasound (5) due to its excellent soft tissue contrast, lack of ionizing radiation exposure, and well-validated accuracy and reliability for blood flow measurements.

Cardiac MRI technology has made tremendous advances within the last two decades. The continued improvements in the MRI hardware performance have enabled widespread use of steady-state free precession (SSFP) sequences (6-7), which provides a high signal and excellent contrast between blood and myocardium. The development of T2-Prep has allowed for further enhancement of the blood-myocardium contrast (8). Additionally, phase-contrast MRI is now a well-established technique for evaluating blood flow in the great vessels. Despite the technical advances, however, the use of MRI for fetal cardiac imaging remains in its infancy. A major problem in adapting technology of adult cardiac MRI to fetal MRI is the lack of ECG or external pulse wave trigger signal for the fetus, which is usually required for high quality cardiac imaging.

Cardiac self-gating is a type of motion compensation method where the cardiac motion gating is based on acquired MRI data instead of ECG. Cardiac self-gating has been previously investigated mostly on adults (9-11). Acoustic gating is an alternative approach (12). Ultrasound gating methods have been previously proposed (13-14) for 3D fetal ultrasound. The few recent studies of cardiac self-gating in the context of fetal cardiac MRI were based on methods previously proposed for adults (15) or based on retrospective analysis of certain imaging artifacts metrics (16). However, fetal cardiac self-gating is more challenging compared to adults due to the smaller size of the fetal heart, and strong interference from maternal signal. Further technical developments specifically for fetal cardiac imaging applications are therefore highly desirable. ECG trigger generally works well on adults; however, cardiac self-gating appears to be one of the few, if not the only, practical solution(s) for obtaining a trigger signal in fetal cardiac MRI. Here, methodologies are developed to accurately and reliably provide a cardiac trigger signal that can be used in virtually all of the fetal cardiac MRI sequences. The successful development of this technology will eliminate a major impediment of fetal cardiac MRI, and will hence bring fetal cardiac MRI closer to clinical practice as a much needed prenatal diagnostic tool for CHD that is complimentary to ultrasound.

Methods disclosed herein are used to reliably provide a cardiac motion self-gating signal for use in fetal cardiac MRI. The developed techniques will then be evaluated on a cohort of pregnant patients who are referred to fetal echocardiography for suspected CHD of the fetus.

Several approaches are taken to refine the ability to derive cardiac self-gating signal in the context of fetal cardiac MRI.

The efficacy of the optimized methodology in fetal cardiac MRI will be evaluated. The image quality and diagnostic value of fetal cardiac images acquired will be subjectively and quantitatively evaluated using the proposed prospective cardiac self-gating trigger signal. They will also be compared the MR-based diagnosis with fetal ultrasound.

Pregnant female patients will be recruited for these experiments. In some embodiments, 20 or more pregnant female patients in the second or third trimester who are referred to fetal echocardiography for suspected CHD will be recruited. In some embodiments, 40 or more such pregnant female patients will be recruited. In some embodiments, 50 or more such pregnant female patients will be recruited. In some embodiments, 60 or more such pregnant female patients will be recruited. In some embodiments, 80 or more such pregnant female patients will be recruited. In some embodiments, 100 or more such pregnant female patients will be recruited. In some embodiments, 120 or more such pregnant female patients will be recruited. In some embodiments, 150 or more such pregnant female patients will be recruited. In some embodiments, 200 or more such pregnant female patients will be recruited.

In some embodiments, the patients will be enrolled in two different groups. For example, the patients can be separated into group A (including those whose echocardiography examinations are adequate) and group B (including those whose echocardiography are not). For example, among 40 patients, 25 can be enrolled in Group A while 15 can be enrolled in Group B.

In some embodiments, the patients will be advised to fast for 4 hours before imaging and to empty her bladder immediately before scanning. For patients in both groups, the following sequences will be performed: 1) 2D multi-slice SSFP cine cardiac MRI in both short and long axis with and without the proposed cardiac self-gating technology; 2) A 2D multi-slice segmented TSE sequence with and without cardiac self-gating in the coronal orientation covering the fetal heart, pulmonary arteries, and aorta. The most relevant sequence parameters for cine cardiac MRI will be: TR/TE=3.5/1.7 ms, voxel size=1.3×1.3 mm, slice thickness=3 mm, flip angle=60°, 15 cardiac phases, GRAPPA=2, maternal breath-hold time=15 s. The relevant sequence parameters for the TSE sequence include: TR=70 ms, echo spacing=8 ms, voxel size=1.3×1.3 mm, slice thickness=3 mm, flip angle=90°, no parallel imaging, maternal breath-hold time=15 s. The sequences will be repeated in case fetal bulk motion causes obvious motion artifacts/ghosting/blurring in the images or if bulk fetal motion is detected by the self-gating MOCCA echoes.

Possible indications for the patients will include the following: group A, suspected case of heterotaxy, pulmonary artery/vein abnormalities, systemic venous abnormalities, aortic arch anomalies, and functional ventricular function abnormalities; and for group B, limited evaluation of fetal echocardiographic anatomy/function secondary to fetal lie, multiple gestation, maternal habitus, oligohydramnios, and other technical factors. The image slice orientation will be changed appropriately based on the specific indication of the patient.

Data Analysis:

For each sequence, the two acquisitions (with and without cardiac self-gating) will be assigned subjective scores on a 1-4 scale and the scores will be compared using Wilcoxon signed rank test. The blood-myocardium border sharpness will be quantified (21) and compared using paired t-test. The hypothesis to test is the self-gated images will have better sharpness and subjective quality scores. A second hypothesis is that the cardiac self-gated fetal MRI will have good agreement with findings from echocardiography. To test this, the MRI images in group A will be evaluated by blinded experienced evaluators. The diagnosis based on MRI will then be compared with echocardiography and a match in diagnosis will be determined by consensus of the evaluators.

A chi-square test for correlated proportions will be used. If correct diagnosis of 80% with echocardiography and 90% with MRI is assumed, the sample size of 40 with a 5% level of significance would yield 85% power.

Having described the invention in detail, it will be apparent that modifications, variations, and equivalent embodiments are possible without departing the scope of the invention defined in the appended claims. Furthermore, it should be appreciated that all examples in the present disclosure are provided as non-limiting examples.

### REFERENCES

1. Roger V L, et al., Heart disease and stroke statistics—2011 update: a report from the American Heart Association. Circulation 2011; 123 (4):e18-e209.
2. Sklansky M S, et al., Real-time three-dimensional fetal echocardiography: initial feasibility study. J Ultrasound Med 1999; 18(11):745-752.
3. Hendler I, et al., The impact of maternal obesity on midtrimester sonographic visualization of fetal cardiac and craniospinal structures. Int J Obes Relat Metab Disord 2004; 28(12):1607-1611.
4. Bromley B, et al., Fetal echocardiography: accuracy and limitations in a population at high and low risk for heart defects. Am J Obstet Gynecol 1992; 166(5):1473-1481.
5. Saleem S N. Feasibility of MRI of the fetal heart with balanced steady-state free precession sequence along fetal body and cardiac planes. AJR Am J Roentgenol 2008; 191(4):1208-1215.
6. Deimling M, Heid O. Magnetization prepared trueFISP imaging; 1994; San Francisco. p 495.
7. Bi X, et al., Reduction of flow- and eddy-currents-induced image artifacts in coronary magnetic resonance angiography using a linear centric-encoding SSFP sequence. Magn Reson Imaging 2007; 25(8):1138-1147.
8. Brittain J H, et al., Coronary angiography with magnetization-prepared T2 contrast. Magn Reson Med 1995; 33(5):689-696.
9. Larson A C, et al., Self-gated cardiac cine MRI. Magn Reson Med 2004; 51(1):93-102.
10. Crowe M E, et al., Automated rectilinear self-gated cardiac cine imaging. Magn Reson Med 2004; 52(4):782-788.
11. Buehrer M, et al., Prospective self-gating for simultaneous compensation of cardiac and respiratory motion. Magn Reson Med 2008; 60(3):683-690.
12. Frauenrath T, et al., Feasibility of cardiac gating free of interference with electro-magnetic fields at 1.5 Tesla, 3.0 Tesla and 7.0 Tesla using an MR-stethoscope. Invest Radiol 2009; 44(9):539-547.
13. Sklansky M S, et al., Usefulness of gated three-dimensional fetal echocardiography to reconstruct and display structures not visualized with two-dimensional imaging. Am J Cardiol 1997; 80(5):665-668.
14. Nelson T R, et al., Three-dimensional echocardiographic evaluation of fetal heart anatomy and function: acquisition, analysis, and display. J Ultrasound Med 1996; 15(1):1-9 quiz 11-12.
15. Yamamura J, et al., Self-gating MR imaging of the fetal heart: comparison with real cardiac triggering. Eur Radiol 2011; 21(1):142-149.
16. Jansz M S, et al., Metric optimized gating for fetal cardiac MRI. Magn Reson Med 2010; 64(5):1304-1314.
17. Bieri O, et al., Analysis and compensation of eddy currents in balanced SSFP. Magn Reson Med 2005; 54(1):129-137.
18. Brau A C, Brittain J H. Generalized self-navigated motion detection technique: Preliminary investigation in abdominal imaging. Magn Reson Med 2006; 55(2):263-270.
19. White R D, et al., Electrocardiograph-independent, “wireless” cardiovascular cine MR imaging. J Magn Reson Imaging 1991; 1(3):347-355.
20. Spraggins T A. Wireless retrospective gating: application to cine cardiac imaging. Magn Reson Imaging 1990; 8(6):675-681.
21. Hu P, et al., Motion correction using coil arrays (MOCCA) for free-breathing cardiac cine MRI. Magnetic Resonance in Medicine 2011:In Press, Reprint enclosed in Appendix.
22. Nam J M. Establishing equivalence of two treatments and sample size requirements in matched-pairs design. Biometrics 1997; 53(4):1422-1430.
23. Deklerck RaS, P. et al., An ECG trigger module for the acquisition of cardiac MR images. 1994; Bethesda, Md., USA. p 533-536.
24. Damji A A, et al., RF interference suppression in a cardiac synchronization system operating in a high magnetic field NMR imaging system. Magn Reson Imaging 1988; 6(6):637-640.
25. Rokey R, et al., Monitoring of acutely ill patients during nuclear magnetic resonance imaging: use of a time-varying filter electrocardiographic gating device to reduce gradient artifacts. Magn Reson Med 1988; 6(2):240-245.
26. Tenforde T S. Magnetically induced electric fields and currents in the circulatory system. Prog Biophys Mol Biol 2005; 87 (2-3):279-288.
27. Wendt R E, et al., Electrocardiographic gating and monitoring in NMR imaging. Magn Reson Imaging 1988; 6(1):89-95.
28. Wielandner A, et al., Potential of magnetic resonance for imaging the fetal heart. Semin Fetal Neonatal Med 2013.
29. Peters M, et al., Monitoring the fetal heart non-invasively: a review of methods. J Perinat Med 2001; 29(5):408-416.
30. Manganaro L, et al., Potential role of fetal cardiac evaluation with magnetic resonance imaging: preliminary experience. Prenat Diagn 2008; 28(2):148-156.
31. Roy C W, et al., Dynamic imaging of the fetal heart using metric optimized gating. Magn Reson Med 2013.
32. Hiba B, et al., Cardiac and respiratory double self-gated cine MRI in the mouse at 7 T. Magn Reson Med 2006; 55(3):506-513.
33. Hiba B, et al., Cardiac and respiratory self-gated cine MRI in the mouse: comparison between radial and rectilinear techniques at 7 T. Magn Reson Med 2007; 58(4):745-753.
34. Bammer R, et al., Augmented generalized SENSE reconstruction to correct for rigid body motion. Magn Reson Med 2007; 57(1):90-102.
35. Bydder M, et al., SMASH navigators. Magn Reson Med 2003; 49(3):493-500.
36. Offerman E J, et al., Prospective self-gated nonenhanced magnetic resonance angiography of the peripheral arteries. Magn Reson Med 2013; 69(1):158-62.
37. Spincemaille P, et al., Kalman filtering for real-time navigator processing. Magn Reson Med 2008; 60(1):158-68.
38. Liu J, et al., Respiratory and cardiac self-gated free-breathing cardiac CINE imaging with multiecho 3D hybrid radial SSFP acquisition. Magn Reson Med 2010; 63(5):1230-7.
39. Kolbitsch, C., et al., (2013), Cardiac functional assessment without electrocardiogram using physiological self-navigation. Magn Reson Med. doi: 10.1002/mrm.24735

### EXAMPLES

The following non-limiting examples are provided to further illustrate embodiments of the invention disclosed herein. It should be appreciated by those of skill in the art that the techniques disclosed in the examples that follow represent approaches that have been found to function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

### Example 1

### Validation Analysis with ECG Gating

Preliminary experiments were performed here on two healthy adults. A breath-held steady-state free precession (SSFP) cardiac cine MRI sequence with retrospective ECG triggering that has been used clinically was selected. The sequence was modified and used to acquire an additional non-PE k-space center line before each k-space segment for every cardiac phase.

The modified sequence was performed on the volunteers and cardiac self-gating signal was derived using the following two methods. In the first method, similar to what Larson et al., and Crowe et al. proposed (9-10), only the magnitude of k-space center sample from the coil with highest amplitude was used as the self-gating signal. In the second method, a column vector (i.e., MOCCA echo) was constructed by “stacking up” the magnitude of the acquired non-phase-encoded center lines from multiple coils, similar to what was used in a recent publication on MOCCA respiratory self-gating (21). The first MOCCA echo was initially chosen as the MOCCA echo reference and the complex difference between subsequent MOCCA echoes and MOCCA echo reference was calculated. The L-2 norms of the complex differences were used as the cardiac self-gating signal. No filters were used on the signal and the self-gating trigger was retrospectively identified by thresholding the self-gating signal (9). Once a trigger is identified, the MOCCA echo corresponding to the trigger time point will be set as the new MOCCA echo reference, which was subsequently used during the next heart cycle (FIG. 6). To study the effect of eddy current and “stepping” PE gradients, the same sequence was performed, but with the PE gradients turned off to eliminate the effects of eddy currents caused by “stepping” PE gradients.

Subsequently, the aforementioned self-gating algorithms were applied on the new non-phase-encoded data set. FIG. 1 shows a typical comparison of self-gating signals with the PE gradients turned on and off. In this example, the conventional methods in FIG. 3a provided rather noisy self-gating signal with various significant artifacts. It might be possible to derive a cardiac trigger signal from this data, but the signal quality would not be sufficient for providing reliable trigger signal for routine clinical use.

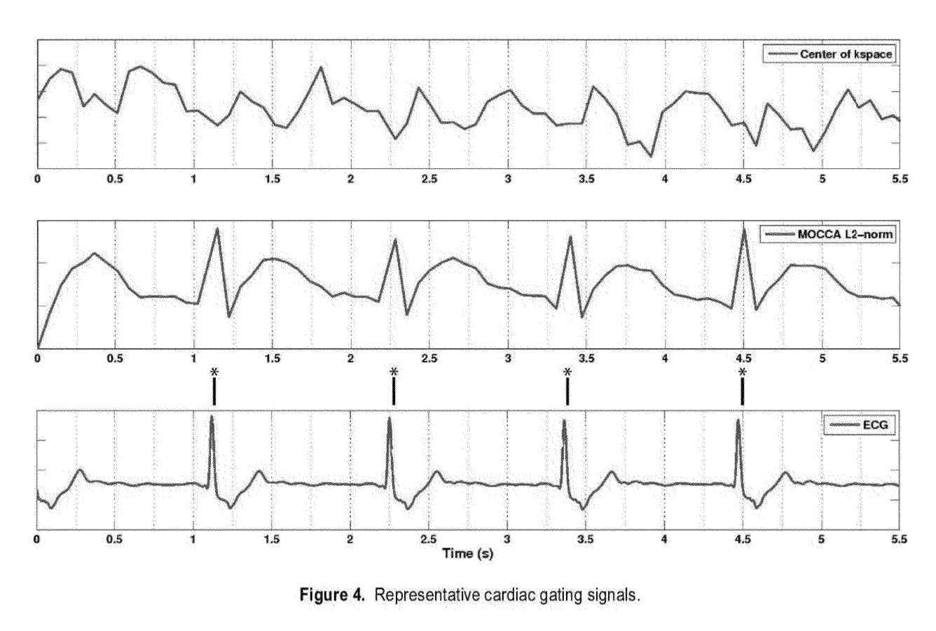

The preliminary results were acquired on healthy adult subjects. Greater distortion of fetal cardiac self-gating signal is expected. The self-gating signal with PE gradients turned off (FIG. 3b) is of much higher quality with less noise and distortions. One of the problems with existing methods is that, compared to short axis view, they are much less reliable for generating self-gating trigger signal in other slice orientations where the in-slice blood volume change is not as dramatic, such as four-chamber view (9). In the example shown in FIG. 4, the method based on center of k-space signal fails to provide a useable signal in the four-chamber view, whereas MOCCA provides excellent trigger signal. One of problems with existing methods is the significant variation in the morphology of the signal depending on the subject and the slice orientation (9). MOCCA has clear advantage in this regard. The trigger position detected by MOCCA correspond precisely to the ECG R wave in both four-chamber and short axis (data not shown) views. The MOCCA retrospectively self-gated images were identical to the ECG gated images due to accurate trigger detection (data not shown).

### Example 2

### Quantitative Evaluation Analysis with ECG Gating

Methods:

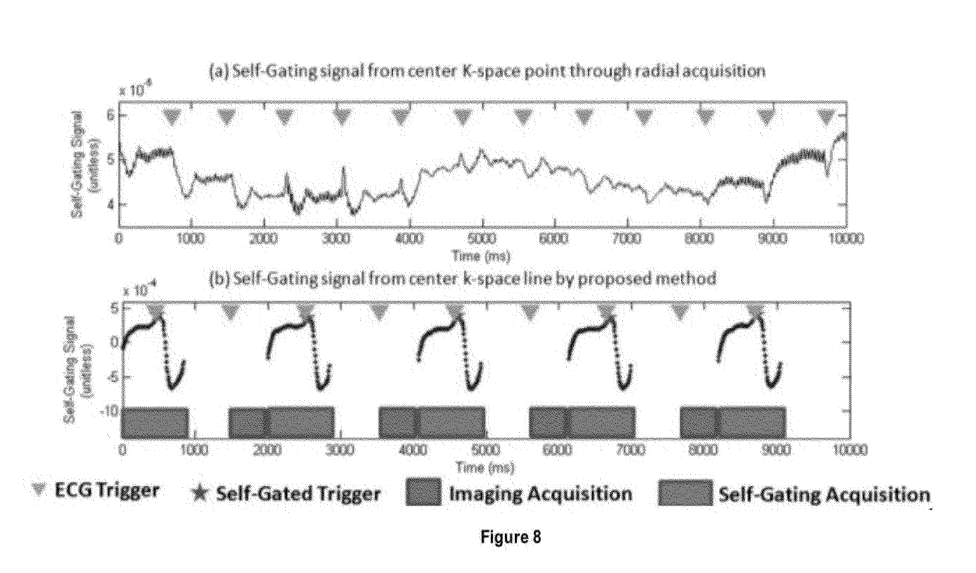

Conventional cardiac self-gating uses the k-space center from a radial acquisition to represent the cardiac motion. However, the acquired motion signal by this method suffers from drifting and distortion shown in FIG. 8a, making it difficult to derive reliable cardiac triggers. The hypothesis is that since the cardiac motion signal acquisition was combined with the imaging acquisition, it is modulated by the eddy currents from the varying phase-encoding (PE) or radial acquisition gradients during imaging.

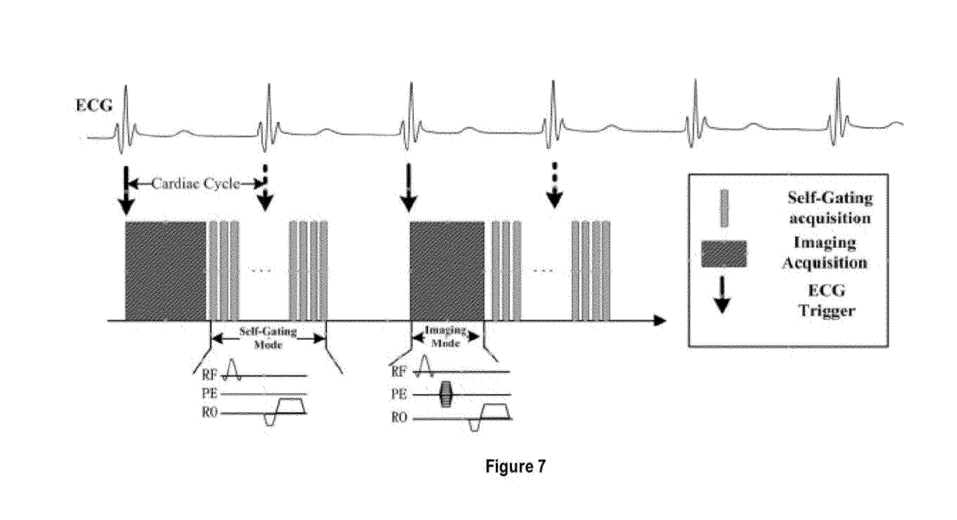

To reduce the signal interference associated with existing self-gating techniques, a self-gating approach was proposed where the data acquisition switches between imaging mode and self-gating mode as shown in FIG. 7. A custom prospectively ECG triggered cardiac cine pulse sequence was implemented by adding multiple dedicated self-gating acquisitions at the end of each imaging window. During the self-gating mode, the pulse sequence is the same as the imaging mode except the phase-encoding gradient is turned off so that the center k-space line is repeatedly acquired. To validate the self-gating approach and compare with the ground truth ECG triggers, the sequence is prospectively triggered by ECG for every two heartbeats and the self-gating mode duration was set long enough to cover the ECG R wave of every other heartbeat so that the calculated self-gating triggers can be verified against the corresponding ECG R wave (FIG. 7). The custom cardiac cine sequence was performed on 4 healthy volunteers with 22 total breath-held cine scans to cover different slice orientations. The self-gating raw data was exported offline for processing and real-time ECG signal and trigger was recorded as the ground truth.

Principle Component Analysis (PCA) was used to extract the cardiac motion signal from the acquired data. Trigger is then detected by finding the local maximum with an adaptive threshold. As a comparison, the k-space center point (instead of the full k-space center line) from the acquired self-gating data was used to generate a self-gating trigger signal based on previously described method.

Result:

FIG. 8b shows that the cardiac self-gating triggers generated by the proposed method matches the corresponding ECG R wave. Based on data from all 22 scans, a total number of 122 self-gating triggers were detected with 100% trigger detection rate. Quantitative evaluation result in Table.1 including mean trigger delay1 (i.e., the delay between the ECG R wave trigger and the self-gating triggers) and mean temporal variability1 (i.e., standard deviation of trigger delay for each acquisition) indicates the proposed method offers accurate and robust cardiac triggers. However, using previous methods on the k-space center point only, 65% of the triggers in all 12 scans from the same 4 subjects were detected.

Discussion:

The purpose of this study is to verify that a self-gating acquisition using non-phase-encoded k-space lines center that is separate from the imaging data acquisition is capable of deriving more precise and robust cardiac motion triggers.

The same sequence framework and algorithm can be used in implementing a ECG-free, completely self-triggered sequence. The sequence switches from self-gating mode, where the PE gradients are turned off, to imaging mode as soon as a new self-gating trigger is detected and switches back after imaging acquisition to detect the next trigger. Such implementation requires real-time trigger detection with minimum processing delay. The PCA technique used is a powerful tool to extract the cardiac motion while suppressing other non-cardiac motion and noises. Using such technique, the trigger could be detected without a high-order frequency filter which is often required by other self-gating method2 and causing inevitable and significant processing delay. To summarize, the method differs from other cardiac self-gating techniques in four aspects: 1) The entire k-space center line is used instead of the center point; 2) Coil arrays were used instead of a single coil3; 3) The self-gating signal is derived from repeatedly acquired non-phase-encoded k-space center line and is therefore free of aforementioned signal interference. 4) PCA is used to further reduce any residual interference and enabled real-time trigger detection. The technique disclosed herein is able to achieve 100% detection rate with <5 ms temporal variability. Furthermore, it ensures a reliably detection of the onset of the ventricular contraction 20-50 ms after ECG R wave, which has not been achieved using previous methods.

Conclusion:

The data demonstrates that the proposed method can offer cardiac motion self-gating signal that is free of distortion or artifacts usually seen in traditional method and therefore improve cardiac trigger detection accuracy and reliability. Future work will be focused on implementing it in a sequence for real time prospectively cardiac self-gated MRI.

TABLE 1
Quantitative evaluation of the detected selfgating
triggers using ECG as reference

| | | |
|---|---|---|
| | Vertical | Horizontal |
| Short Axis | Long Axis | Long Axis |

| | | | | | | |
|---|---|---|---|---|---|---|
| Mean Delay | 17.9 | ms | 29.1 | ms | 58.1 | ms |
| Temporal Variability | ±4.3 | ms | ±4.7 | ms | ±3.8 | ms |

### Example 3

### Improved Cardiac Motion Self-Gating

Background:

Cardiac motion self-gating is a technique where MRI signal is used to derive motion triggers instead of ECG, which might be problematic in high BO field or cases where ECG is not accessible (e.g., fetal cardiac imaging). However, the performance of existing cardiac self-gating approaches has not yet enabled clinical utility. A novel cardiac self-gating strategy was proposed and evaluated, which potentially improves the trigger detection accuracy and reliability.

Methods:

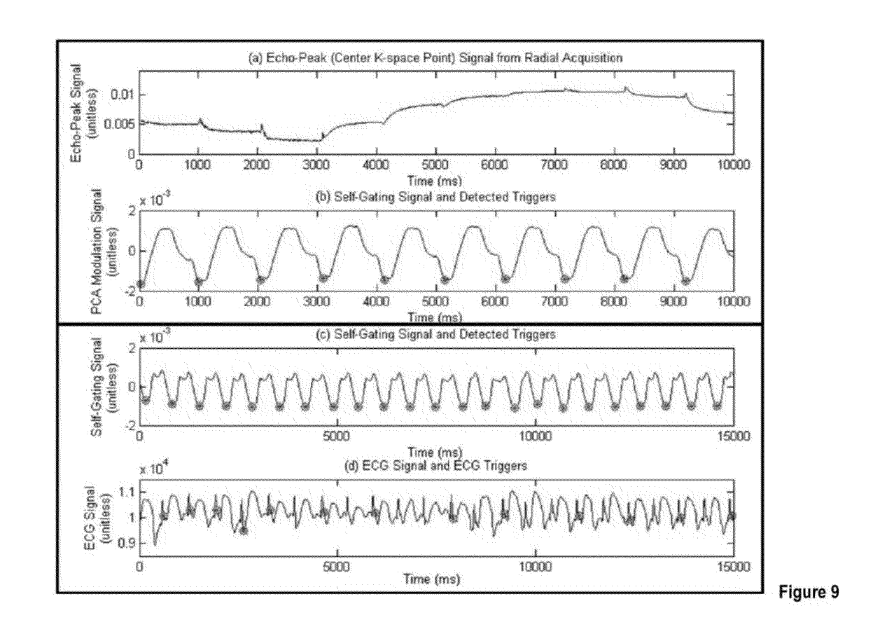

Conventional cardiac self-gating uses the k-space center from a radial acquisition to represent the cardiac motion and derive triggers. However, this strategy suffers from signal drifting and distortion shown in FIG. 9a. This is possibly due to the fact that the k-space center signal was modulated by the eddy currents from the varying phase-encoding (PE) or radial acquisition gradients. Such interferences should be removed for robust self-gating. To test this hypothesis, a Cartesian breath-held cardiac cine sequence was run with phase-encoding gradient turned off. Principle Component Analysis (PCA) was used to extract the cardiac motion signal from the acquired data. Trigger is then detected by finding the local maximum with an adaptive threshold. The method differs from other cardiac self-gating techniques in four aspects: 1) The whole k-space center line is used instead of the center point only; 2) Coil arrays were used instead of a single coil; 3) The self-gating signal is derived from repeatedly acquired non-phase-encoded k-space center line and is therefore free of aforementioned signal interference. 4) PCA is used to further reduce any residual interference.

FIG. 7 shows a potential implementation in a cardiac MRI sequence. It consists of a self-gating mode where the k-space center line is repeatedly acquired and an imaging mode where k-space is sampled. The sequence switches from self-gating mode, where the PE gradients are turned off, to imaging mode when a new trigger is detected and switches back after imaging to wait for the next trigger.

Results:

FIG. 9b shows the cardiac self-gating signal and trigger generated by the proposed method on the same subject for FIG. 9a. FIGS. 9c and 9d show the result from a 3 T scanner where the quality of ECG is poor while the self-gating method could still provide accurate triggers. Based on data from 8 healthy volunteers, the overall trigger detection rate was 99% (one failed due to non-ideal breathholding) and the average temporal variability of triggers was ±7.79 ms using the ECG as reference.

On 3 subjects using the k-space center point only as previously described, the overall detection rate was only 65%.

Conclusion:

The data demonstrates that the proposed cardiac self-gating method can significantly reduce the drift and distortion of the self-gating signal and therefore improve cardiac trigger detection accuracy and reliability. Future work will be focused on implementing the technique in an imaging sequence as FIG. 7.

### Example 4

### Improved Cardiac Imaging

The self-gating mode was first run alone to compare the detected self-gating trigger with the recorded ECG trigger. Data were acquired from 10 healthy volunteers using a Cartesian breath-held cardiac CINE sequence with phase-encoding gradient turned off. Two quantitative measurement to evaluate the detected self-gating trigger are defined as:

MeanDelay=mean(sgTrigger−ecgTrigger)

Temporal Variability=RMS(sgTrigger−ecgTrigger)

The proposed method was then fully implemented on a Siemens System. It consists of a pulse sequence (FIG. 1) installed on the scanner to acquire the self-gating signal and a program containing the proposed self-gating algorithm installed on the image reconstruction system to provide real-time feedback to the sequence. Self-gated cardiac CINE images were acquired on 4 volunteer volunteers with 2 orientations (short axis, horizontal long axis). Standard ECG-gated CINE images were also acquired on each volunteer for comparison.

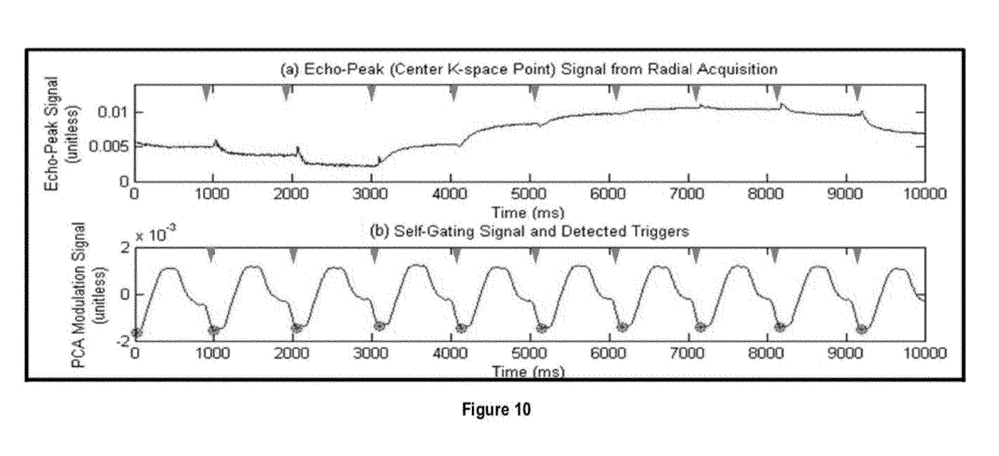

FIG. 10 shows the result of validation on “self-gating mode” alone. FIG. 10a depicts the self-gating signal acquired using method in Larson et al and FIG. 10a depicts is the self-gating signal and triggers (marked *) by the proposed method. The signal generated by the proposed method is free of the drifting and distortion seen in FIG. 10a and the detected self-gating trigger perfectly matches the corresponding ECG R wave (marked by ▾). Quantitative evaluation result in Table 2 further proves that the proposed method offers accurate and robust cardiac triggers.

TABLE 2
Quantitative Evaluation of the self-gating
triggers using recorded ECG as reference

| | | | Temporal |
|---|---|---|---|
| | Detection Rate | Mean Delay | Variability |
| Short Axis | 100% | 17.9 ms | 4.3 ms |
| VLA | 100% | 29.1 ms | 4.7 ms |
| HLA | 98% | 58.1 ms | 3.8 ms |

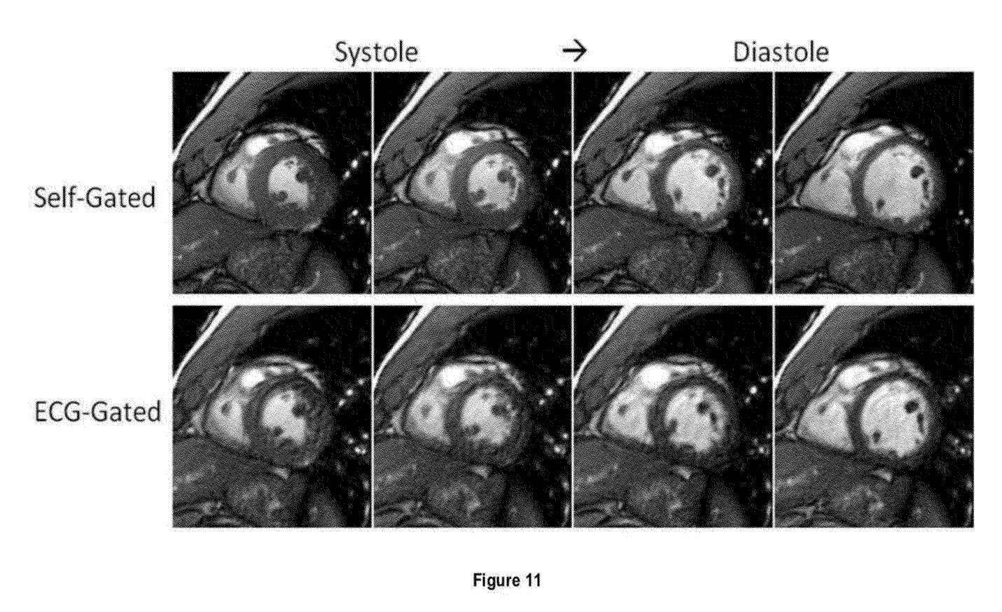

FIG. 11 shows 3 T cardiac CINE images (4 out of 17 selected cardiac phase) acquired by the proposed self-gating method alone with a separate standard ECG-gated CINE image of the same subject as comparison. The ECG signal was degraded resulting in inferior CINE images, whereas the self-gating was able to accurately gate the cardiac motion.

The data shows that the proposed method is capable of offer cardiac self-gating triggers with high accuracy and reliability. The images acquired by the proposed method has equivalent quality with the ones acquired by standard ECG-gated sequence, meaning that the proposed self-gating method could potentially become a replacement of conventional ECG-gated cardiac sequence.

### Example 5

### Cardiac Motion Self-Gating

### Online Prospective Case Study

Purpose:

To develop a prospective cardiac motion self-gating method that provides robust and accurate cardiac triggers in real time.

Methods:

The proposed self-gating method consists of an “imaging mode” that acquires the k-space segments and a “self-gating mode” that captures the cardiac motion by repeatedly sampling the k-space centerline. A training based principal component analysis algorithm is utilized to process the self-gating data where the projection onto the first principal component was used as the self-gating signal. Retrospective studies using a sequence with self-gating mode only was performed on 8 healthy subjects to validate the accuracy and reliability of the self-gating triggers. Prospective studies using both ECG-gated and self-gated cardiac CINE sequences were conducted on 6 healthy subjects to compare the image quality.

Results:

Using the ECG as the reference, the proposed method was able to detect self-gating triggers within ±10 ms accuracy on all 8 subjects in the retrospective study. The prospectively self-gated CINE sequence successfully detected 100% of the cardiac triggers and provided excellent CINE image quality without using ECG signals.

Conclusion:

The proposed cardiac self-gating method is a robust and accurate alternative to conventional ECG-based gating method for a number of cardiac MRI applications.

In many cardiac magnetic resonance imaging (CMR) applications, the data acquisition needs to be synchronized with the cardiac motion. Typically, electrocardiogram (ECG) is used to monitor the cardiac motion and control the timing of data acquisition. This is commonly referred as ECG gating or ECG triggering. For a normal ECG signal, the QRS complex has the highest amplitude peak and sharpest upstroke, which is often used as cardiac triggers (23). However, the ECG based cardiac gating is associated with several potential issues. First, the ECG signal is sometimes interfered by the time varying magnetic field of the MRI system. Such interferences can be severe in higher fields and eventually cause degraded image quality due to synchronization errors (24-27). Furthermore, there are applications when ECG signal is difficult to acquire or even inaccessible, such as fetal cardiac imaging (28, 29). As an alternative to ECG, self-gating uses intrinsic MRI signal to detect cardiac motion and synchronize the timing of imaging events. It provides direct measurement of the mechanical motion instead of the electrical signal as is the case with ECG, and hence does not suffer from the aforementioned issues of ECG. It is potentially a valuable alternative approach for fetal cardiac motion gating in fetal cardiac MRI (15, 30, 31).

Self-gating techniques normally consist of two parts: acquisition and processing. In the acquisition part, selected k-space data is repeatedly acquired to form the time resolved cardiac motion self-gating signal. Previously reported cardiac self-gating approaches use the k-space center point in a radial (9, 32) or Cartesian (11, 10, 10, 33, 20, 19) sampling trajectory as the self-gating signal. A number of algorithms have been developed to process the self-gating signal, including echo peak modulation, projection-based center of mass and low-resolution region of interest correlation (9, 20, 19). Larson et al., (9) proposed a technique where self-gating signal is derived retrospectively from the k-space center point in a radial sampling trajectory. Cardiac triggers are generated by finding the peak of the center point signal after a low-pass filter. Previous studies by Hu et al., (21) on Motion Correction using Multiple Coil Array (MOCCA) suggests that redundant data by coil arrays could provide richer information to estimate and correct motion (34, 35). A MOCCA echo is formed by concatenating the k-space centerlines acquired by coil arrays into a single vector. The advantage of using a MOCCA echo in self-gating is that the motion information is greatly enriched without the need of additional acquisition time. Although the MOCCA technique is originally designed for respiratory motion gating, its principle is also applicable to cardiac motion. However, a more sophisticated and robust processing algorithm is required to fully exploit the abundant information of MOCCA echoes. In most cardiac self-gating techniques, the cardiac triggers are either generated offline after the acquisition (9, 10) or online during the acquisition (11, 36). Offline gating usually requires a sufficient amount of temporal oversampling and therefore suffers from longer acquisition time. Online self-gating is more efficient because the acquisition of k-space segments is controlled on the fly to make sure sufficient k-space segments are acquired within minimal time. However, it is technically more challenging because of the requirement of deriving self-gating signal and detecting self-gating triggers in real time (37). Despite a number of recent advances, cardiac motion self-gating has not been used in clinical practice, mostly due to limited reliability and reproducibility of the self-gating triggers.

The goal of this study was to develop and validate a prospective online cardiac motion self-gating technique. Several technical advances are included to enable accurate and reliable trigger detection in real time while the sequence is running, including separation of self-gating acquisition from imaging acquisition and use of training based Principal Component Analysis (PCA) algorithm on multi-coil self-gating data processing.

### Prospective Self-Gating Sequence

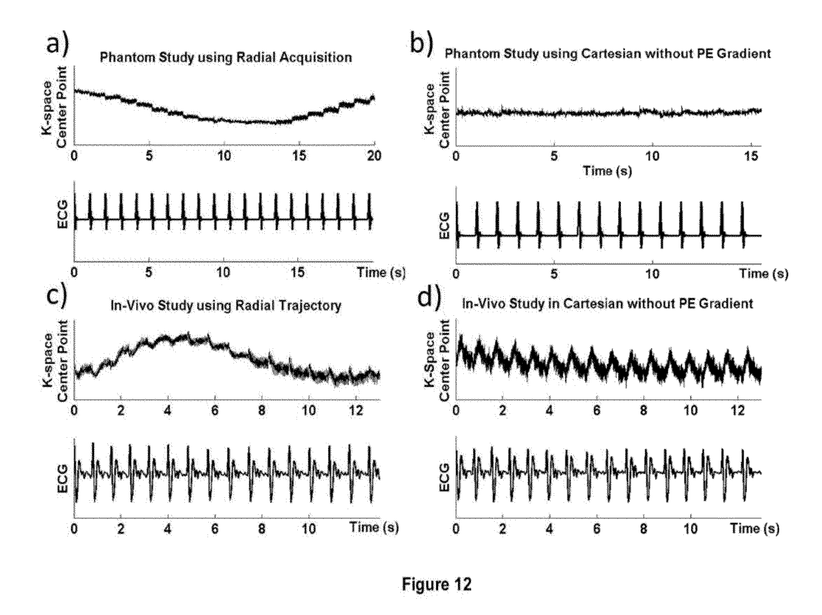

In a conventional self-gating approach, the self-gating signal is typically acquired concurrently with the imaging data, such as using radial sampling where the k-space center point is acquired as part of each radial projection line (9, 32). For Cartesian sampling, several groups have acquired an additional echo or FID signal during the same TR as imaging but immediately before the phase-encoding gradients (10). Additionally, the self-gating data and imaging data can be acquired in an interleaved fashion on a TR to TR basis (19, 20). However, these approaches could suffer from self-gating signal distortions that arise from the history of RF pulses and gradients played before the current TR, and eddy currents generated by the phase-encoding gradients that vary from TR to TR. To test this hypothesis, a radial-based cardiac CINE sequence was run on both a stationary phantom and in-vivo. The ECG signal was recorded for reference during the acquisition (simulated ECG in phantom study). The k-space center point (CP) signal from phantom study (FIG. 12a) has significant drifting. Similar artifacts can also be found in-vivo (FIG. 12c), making it difficult to automatically derive reliable cardiac triggers from the CP signal in real time. A non-phase-encoded Cartesian CINE sequence was run again on the same phantom and human subject. The pulse sequence remains identical in every TR since there is no phase-encoding gradient. CP signal of stationary phantom (FIG. 12b) is free of the aforementioned distortion and the in-vivo CP signal (FIG. 12d) shows clear evidence of cardiac motion, though it is mixed with noise.

Based on data shown in FIG. 12, a two-mode sequence was used to solve the aforementioned self-gating signal distortion problem. Instead of acquiring self-gating and imaging data within the same or successive TRs, the self-gating signal is acquired in a dedicated self-gating acquisition mode that is separated from the image acquisition. The pulse sequence is described in FIG. 1 using cardiac CINE as an example, although the same approach could be extended to other triggered cardiac MRI applications. The sequence starts with a training phase where k-space centerlines are repeatedly acquired for 300 TRs (about 1 second). These data are processed by a PCA training algorithm described in the next section. The purpose of the training is to 1) find the principal component vector that is used to process the multi-dimensional self-gating signal; 2) calculate the threshold for real-time self-gating trigger detection. The self-gating mode starts immediately after the training phase and the PCA projection algorithm is applied to the self-gating data as they are acquired. Upon detection of the self-gating trigger, the sequence immediately switches to imaging mode to acquire the k-space segments. The duration of the imaging mode is set to be shorter than the expected cardiac cycle so that the sequence can switch back to self-gating mode before the next cardiac trigger. Although the sequence switches between the two modes, the only difference in terms of pulse sequence is that the self-gating mode does not use any phase-encoding gradient. All other sequence parameters are maintained, including TR, TE and RF shape and duration. This ensures that the steady state of the magnetization is preserved even during switching, which is very important for the signal quality for both imaging and self-gating. Because the self-gating mode essentially acquires the same k-space centerline repetitively, the self-gating signal distortion problem addressed above is avoided as each new self-gating TR has the same history of RF pulse and gradients, and maintains the same steady state. The acquisitions in the preliminary study using the non-phase-encoded Cartesian CINE sequence (FIG. 12b and FIG. 12d) are essentially the self-gating mode in the proposed sequence. The signal plot shows that the data acquired in the self-gating mode yields much improved self-gating signal quality, which is important for subsequent processing and trigger detection.

### Self-Gating Algorithm

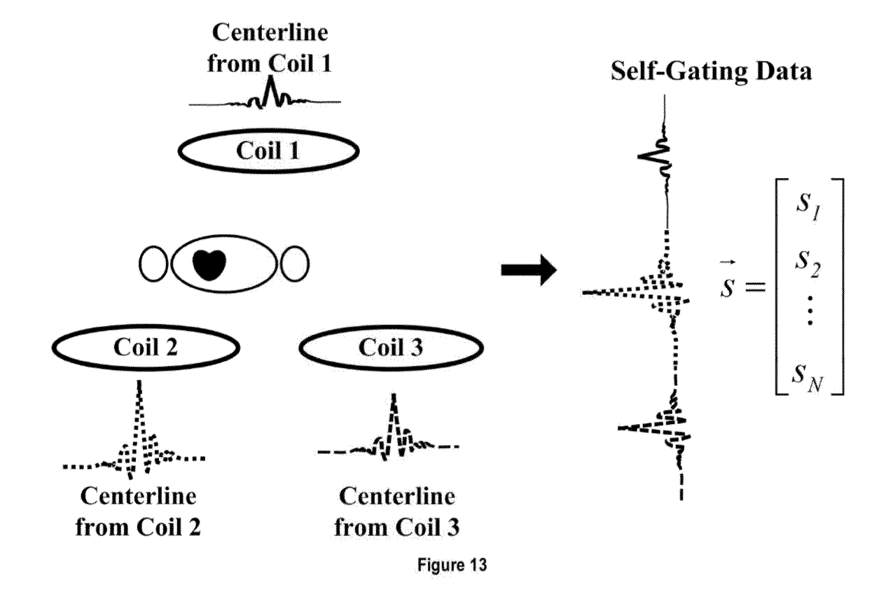

To maximize the available motion information, k-space centerline is acquired using multiple coils rather than k-space center point alone. A MOCCA echo (21) is formed by concatenating the centerline from all coils as shown in FIG. 13. The MOCCA echo, denoted by a vector \vec{S}, is chosen to be the self-gating data. In a typical cardiac MRI sequence, the number of sample in a single k-space centerline ranges from 128 to 512 and up to 18 coils are used for acquisition. As a result, the size of a MOCCA vector could easily reach the order of thousands. Each of the N elements in the MOCCA vector is an independent measurement of cardiac motion because it is modulated by unique k-space positions and coil sensitivity profile (21).

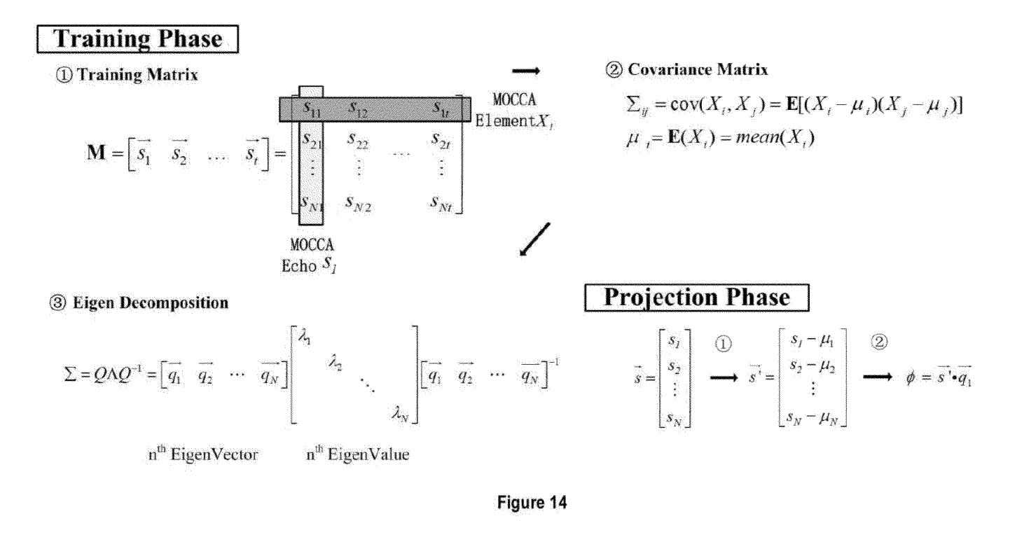

Given the abundant information provided by the MOCCA echo, it is the goal of the self-gating data processing algorithm to combine all measurements in the MOCCA echo in such a way that cardiac motion is enhanced while noise is suppressed. Cardiac motion was assumed to be the most significant factor in causing self-gating signal variance in a breath-held cardiac scan. Therefore, principal component analysis (PCA) algorithm was used in the algorithm because it is a useful data processing technique to represent high dimensional data by their variation significance. For simplified computation and real-time processing, PCA algorithm was implemented in a training-projection fashion as described in FIG. 14. In the training phase, a total number of T=300 MOCCA echoes are collected to construct the training matrix M. Each column in the matrix represents a MOCCA echo from a single self-gating acquisition \vec{s} and each row contains all the measurements of a MOCCA element X. Given the training matrix M, a covariance matrix Σ is derived by calculating the covariance of every two MOCCA element. Then, Eigen-decomposition is performed on the covariance matrix to have the eigenvectors and corresponding eigenvalues. The first eigenvector was referred to as the principal component. This is because the training dataset exhibit maximum variance in that direction, which is assumed to be the result of cardiac motion. Therefore, only the first eigenvector \vec{q₁} is stored for the projection phase.

Compared with the training phase, the calculation of the projection phase is fairly simple. A new MOCCA echo \vec{s} is first “centralized” by subtracting the average value of each MOCCA element. The centralized vector \vec{s′} is then projected onto the principal component direction \vec{q₁} and the projected length is calculated from the dot product of vector \vec{s′} and \vec{q₁}. The scalar φ is the desired cardiac motion measurement from which an accurate and reliable cardiac trigger can be generated.

### Self-Gating Trigger Temporal Variability

In order to validate the proposed self-gating signal acquisition and signal processing strategy, a breath-hold acquisition with self-gating mode was run only by turning off the phase-encoding gradient so that the k-space centerline is repeatedly acquired. 1.5 T Avanto and 3 T Trio (Siemens Healthcare, Erlangen, Germany) scanners were used with a combination of different cardiac orientations, including short axis (SA), vertical long axis (VLA), horizontal long axis (HLA), on 8 healthy volunteers. Other sequence and algorithm parameters include: TR=3.2 ms, TE=1.6 ms, FA=65 training number T=300 for balanced steady state free precession (bSSFP) sequence and TR=6.9 ms, TE=2.4 ms, FA=30, training number T=150 for gradient echo (GRE) sequence. The acquired self-gating data was exported offline and processed by a Matlab (MathWorks, Natick, Mass.) program. Synchronous ECG signal and triggers were recorded with timestamp as the reference. Detection rate (Eq. (1)) and temporal variability (Eq. (2)) were used to assess the reliability and reproducibility of the self-gating (SG) triggers. The temporal variability is calculated as the standard deviation of the time delay between self-gating triggers and corresponding ECG triggers. A smaller temporal variability indicates good temporal consistency between self-gating triggers and ECG triggers. Of note, the ECG monitoring system itself has an inherent systematic variation of up to ±2.5 ms because of its 400 Hz sampling rate.

$$\begin{matrix} R = \frac{number of SG trigger}{number of ECG trigger} & Eq . ( 1 ) \\ {T}_{var} = RMS ( SG - ECG ) = \sqrt{\frac{1}{N - 1} {\sum }_{i = 1}^{N} {( ( SG ( i ) - ECG ( i ) ) - mean ( SG - ECG ) )}^{2}} & Eq . ( 2 )\end{matrix}$$

### In Vivo Prospective Self-Gated Cine MRI

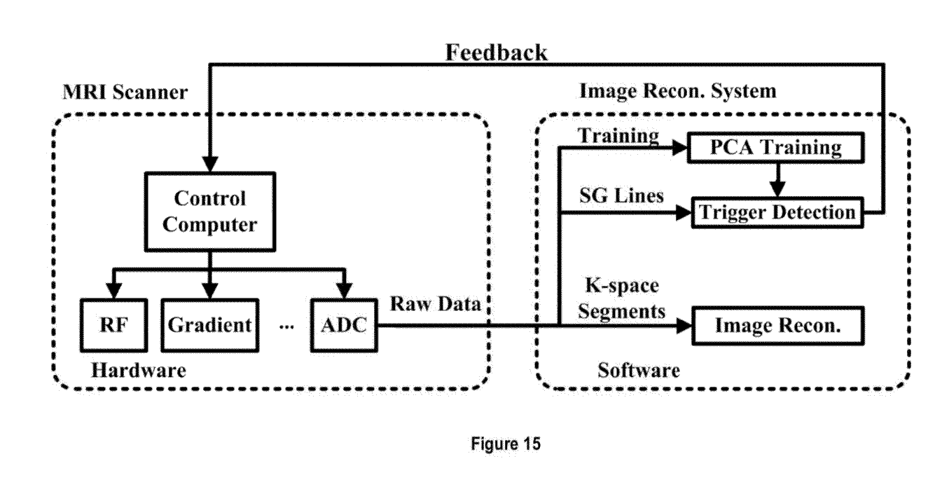

The proposed self-gating acquisition scheme and self-gating algorithm were further implemented in a prospectively self-gated cine sequence. The self-gating data processing algorithm shown in FIG. 15 was developed in Siemens Image Calculation Environment (ICE) using C++ programming language. K-space measurement data from the scanner was sent to the self-gating processing module after each TR with a flag indicating the type of the acquisition (training, self-gating or imaging). The first 299 training data were stored to fill the PCA training matrix. With the arrival of the 300th training data, PCA training program was initiated to find the first principal component of the training matrix as described in FIG. 14. Subsequently, the 300 training data were projected to the principal component direction, resulting in 300 (corresponds to about 1 second) scalar values representing the cardiac motion. An initial cardiac trigger was detected by finding the peak within these measurements. For the successive self-gating data, only PCA projection algorithm was used to calculate the cardiac motion from which cardiac triggers were detected by finding the signal peak that is above the threshold within a sliding window of 5 samples. The threshold was initially defined as 90% of the cardiac trigger during training phase and was updated upon each detected trigger. No filtering was applied before the peak detection due to high quality of self-gating signal. When a self-gating trigger was detected, a feedback signal was immediately sent back to the scanner to stop the current self-gating mode and start the imaging mode. Conventional Fourier based image reconstruction was applied to process the imaging data. In such a way, the sequence switches between self-gating mode and imaging mode until the entire k-space is filled. Immediately after the scan, a series of cardiac CINE image was readily available at the scanner console.

The prospective self-gating sequence was tested on 6 healthy volunteers using the 1.5 T scanner in two orientations (SA and VLA). Real time sequence mode (training, self-gating and imaging) was also recorded as a flag in the raw data. Standard prospective ECG-gated CINE images were also acquired on each volunteer using matched slice orientation as a comparison of image quality. Real-time ECG signal and triggers were recorded for reference, which was used to calculate the temporal variability and detection rate of the prospective data sets according to Eqs. (1) and (2).

### Results

### Self-Gating Trigger Temporal Variability

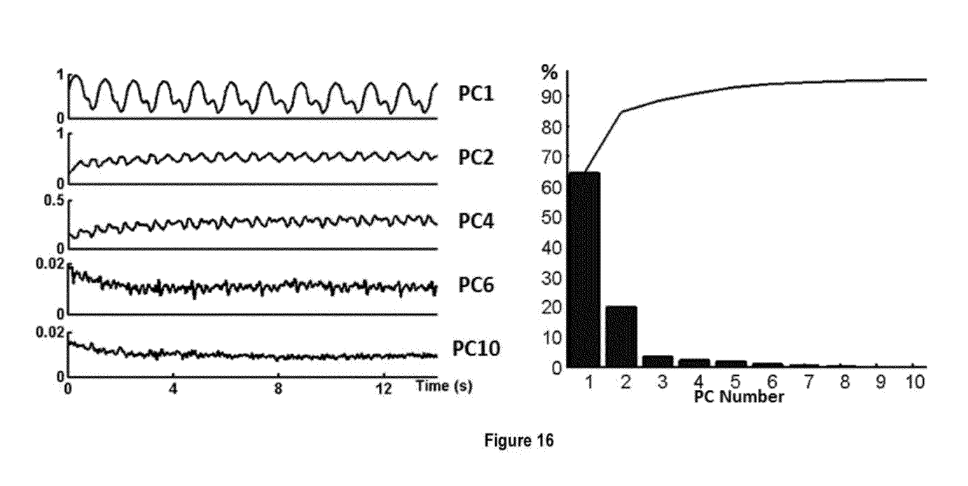

FIG. 16 shows the plot of 5 principal components generated by PCA algorithm from one selected self-gating data as well as their contributions to the total signal variance. The first principal component provide a clear and smooth measurement of cardiac motion while other component are distorted and mixed with noise. Meanwhile, the first component contributes to over 60% of total signal variance, suggesting that most of the motion information in the MOCCA echo is concentrated in the first principal component. Therefore, the first principal component direction was selected to represents the cardiac motion.

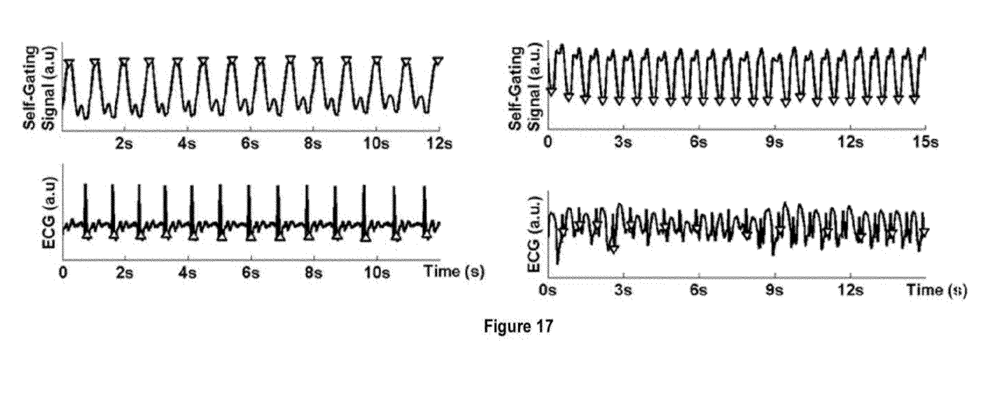

FIG. 17a shows an example of the PCA processed self-gating signal and the corresponding ECG signal from a 1.5 T scanner in cardiac short-axis view. The self-gating signal provided smooth cardiac motion measurement and accurate cardiac triggers that corresponded well to the ECG triggers. FIG. 17b shows another result of the self-gating and ECG signal from a 3 T scanner in a cardiac vertical long axis view. In this particular case, ECG signal was heavily distorted due to interference with varying magnetic field (24-27) during the scan and several ECG triggers were missed by the scanner. However, self-gating signal was capable of providing reliable gating of cardiac motion. Of note, no filter was needed on the self-gating signal.

Table 3 lists the detection rate and temporal variability of the self-gating triggers from 16 experiments in different combination of scanner, sequence and slice orientation. The proposed self-gating method was able to achieve 100% detection rate in most of the experiments with only one exception (#7). In that case, the self-gating signal drifted during the last cardiac cycle so that the threshold-based trigger detection algorithm wasn't able to catch that cardiac trigger. The drifting in this particular case could be caused by respiratory motion due to non-idea breath-hold, which was confirmed with the subject during the experiment. The temporal variability was less than 10 millisecond, suggesting the detected self-gating triggers coincides well with the ECG triggers, though they can be shifted from the QRS complex as shown in FIG. 17.

TABLE 3
Detection Rate and Temporal Variability of Self-Gating Triggers.

| | | | | | Temporal |
|---|---|---|---|---|---|
| # | Scanner | Sequence | View | Det. % | Variability |
| 1 | 1.5 T | GRE | SA | 100% | 9.42 ms |
| 2 | 3.0 T | bSSFP | SA | 100% | 9.94 ms |
| 3 | 1.5 T | GRE | VLA | 100% | 10.1 ms |
| 4 | 3.0 T | GRE | VLA | 100% | 7.77 ms |
| 5 | 1.5 T | GRE | SA | 100% | 9.15 ms |
| 6 | 3.0 T | bSSFP | SA | 100% | 5.75 ms |
| 7 | 1.5 T | GRE | HLA | 93% | 3.36 ms |
| 8 | 3.0 T | bSSFP | HLA | 100% | 4.75 ms |
| 9 | 1.5 T | bSSFP | SA | 100% | 7.24 ms |
| 10 | 3.0 T | bSSFP | SA | 100% | 6.49 ms |
| 11 | 1.5 T | GRE | HLA | 100% | 3.68 ms |
| 12 | 3.0 T | bSSFP | VLA | 100% | 6.67 ms |
| 13 | 1.5 T | GRE | SA | 100% | 5.46 ms |
| 14 | 3.0 T | bSSFP | HLA | 100% | 7.57 ms |
| 15 | 1.5 T | GRE | SA | 100% | 10.0 ms |
| 16 | 3.0 T | bSSFP | VLA | 100% | 2.43 ms |

TABLE 4
statistical result of prospective self-gating sequence.

| | Slice | Detection | Temporal | |
|---|---|---|---|---|
| Subject | Orientation | Rate | Variability | Mean Delay |
| 1 | SA | 100% | 13.9 ms | 236 ms |
| 2 | SA | 100% | 9.1 ms | 222 ms |
| 3 | SA | 100% | 12.1 ms | 228 ms |
| 4 | VLA | 100% | 6.9 ms | 174 ms |
| 5 | VLA | 100% | 13.3 ms | 183 ms |
| 6 | VLA | 100% | 8.4 ms | 176 ms |

### Prospective Self-Gated Cine MRI

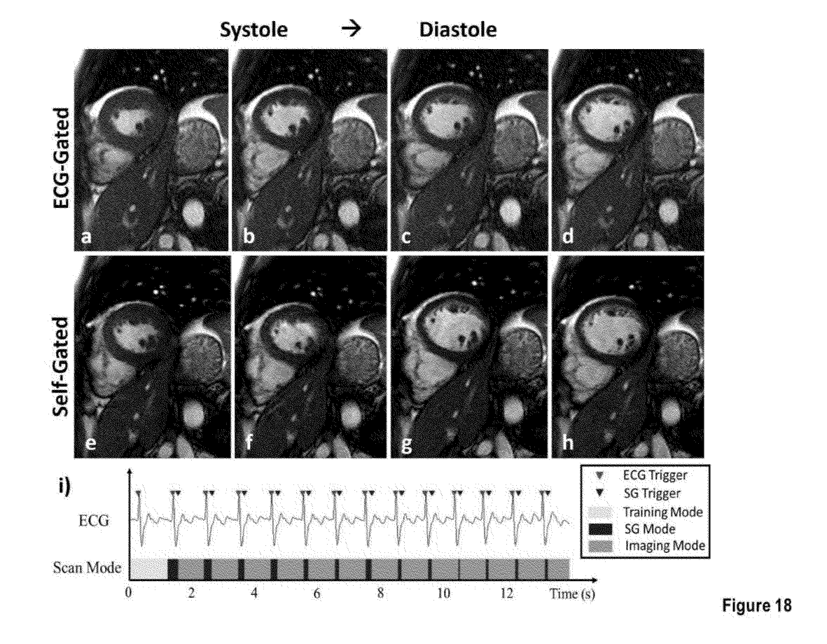

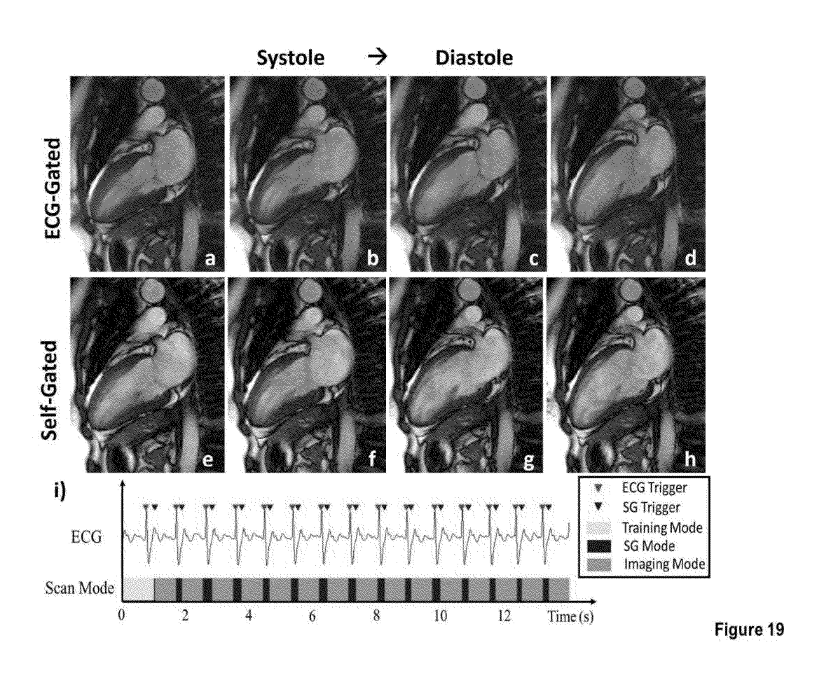

FIG. 18a-h and FIG. 19a-h show selected frames from example CINE images in short-axis and vertical-long-axis views acquired on healthy volunteers using a 1.5 T scanner. There was no noticeable motion artifact in the self-gated images and the overall image quality of self-gated CINE is equivalent with that of ECG-gated. Based on the flags in the raw data, the self-gating trigger was successfully identified in both examples as shown in FIG. 18i and FIG. 19i. There was slight variation in the heart rate during the exam and the duration of the self-gating mode for each heart beat varied accordingly as expected. Table 4 lists the statistical result of all 6 scans. The proposed prospective self-gating method was able to detect 100% of the 85 cardiac triggers over 6 subjects and switch scan mode accordingly. The average temporal variability between self-gating triggers and ECG triggers was 10.6 ms, which was similar to the findings at the temporal variation study. The mean trigger delay when compared with ECG R-wave was approximately 220-230 ms for short axis views and approximately 170-180 ms for vertical long axis views.

A prospective cardiac self-gating technique was introduced and demonstrated in a self-gated cardiac cine sequence that is capable of detecting 100% of the cardiac trigger in real time. The technique is different from other existing self-gating methods in three aspects. First, MOCCA echo (k-space centerline with coil arrays) is used as self-gating data that could provide abundant motion information. Second, the self-gating data is processed by PCA algorithm in a training-projection scheme. Third, a two-mode sequence structure is adopted in which dedicated self-gating acquisitions are separated from the normal imaging acquisition. The proposed technique was evaluated by comparing the self-gating triggers with ECG triggers and the results indicate good temporal consistency between the two. The self-gating technique was further tested in a prospectively self-gated cardiac CINE sequence and showed excellent correspondence of the self-gating triggers to the ECG triggers. The data suggests that this sequence is very reliable in trigger detection and can provide excellent cardiac image quality. The solution uses the clinically available image reconstruction computer to process the self-gating data and send feedback signal to the MRI scanner. Such an implementation is feasible on MRI systems from most major manufacturers without any hardware modification. In this work, the feasibility of the proposed self-gating technique was demonstrated using a self-gated cardiac CINE sequence. Other applications using this self-gating technique have yet to be developed. Some of the examples include, but not limited to self-gated coronary angiography (MRA), cardiac imaging in high magnetic field (7 T and up), and fetal cardiac imaging.

The MOCCA echo used in the proposed self-gating method could better capture cardiac motion than other self-gating data sampling strategy. While k-space center point is only capable to capture the variance of the image DC component and the k-space centerline can further detect the non-DC variance in the k-space readout direction, the MOCCA echo has the intrinsic capability to detect motion in all directions. This is because up to 16 coils are placed in almost every direction around the heart in a conventional cardiac MRI setup. As a result, motion information in any direction could be modulated by individual coil's sensitivity map and reflected in the MOCCA echo. Although a systematic evaluation of the potential of MOCCA echo was not done, the signal quality improvement of FIG. 17 over FIGS. 12b and 12d resulted from the use of MOCCA echo instead of k-space center point.

PCA algorithm can better exploit cardiac motion information provided by MOCCA echo. To address the theory behind the proposed PCA-based algorithm, the task was interpreted as a signal-processing problem in which the desired signal component (i.e., cardiac motion) was enhanced and the unwanted component (i.e., other motion, noise etc.) was suppressed. In such a task, a precise definition of the signal is needed to differentiate it from the noise. Most existing processing algorithms use an explicit definition in image domain to characterize the cardiac motion signal. For example, the method of using the k-space center point defines the cardiac motion as the change of overall image intensity. This is based on the assumption that the variation of blood pool volume is the major contributor of the overall image intensity, which is why some of the existing techniques typically works better at short-axis view because this view is associated with most significant change in blood volume (9). However, the approach appears to work equally well in both short axis and long axis views because the PCA algorithm is not dependent on in-plane blood volume Other algorithms define the cardiac motion by looking for certain features from the Fourier transformed k-space line, including sharp edges, center of mass (COM) etc. Despite the fact that these methods highly depend on specific imaging parameters (e.g., contrast, slice orientation) and the anatomy of individual subjects, they are unable to take advantage of the motion information provided by multiple coils because the processing is done in image domain after combining the signals. On the other hand, the proposed PCA-based algorithm defines the cardiac motion in an implicit way: the cardiac motion is the most significant factors in causing the variance of self-gating signal in a breath-hold cardiac scan. First, this definition is independent of imaging parameters or individual subjects. Second, the processing is performed in k-space signal domain, before combining information from multiple coils and thereby has the potential to take advantage of the MOCCA echo. Third, abundant information in MOCCA echo is better used as all MOCCA channels are combined together in a way to maximize the signal variance. In addition, the proposed PCA algorithm shows good performance in suppressing noise, as shown by the clarity and smoothness of the signal plot in Error! Reference source not found. and FIG. 16 even in the absence of any filtering of the signal.

The proposed PCA algorithm is a training based algorithm. The first 300 self-gating samples are chosen to construct the training matrix. It is because 300 samples take about 1 second (TR=3 ms), which is approximately a completely cardiac cycle. From these training samples, the component with maximum signal variation is found, which is assumed as the cardiac motion component. Therefore, it is desirable that the training period is sufficiently long to cover a complete cardiac motion cycle, but not too long as overall imaging efficiency would decrease. The advantage of such training-based algorithm is that the signal process algorithm is individually tailored for each subject in each scan and no specific parameters is required at the users' end. This is further supported by the data from Table 3 that the same algorithm can be used to process self-gating signals from different scans, on different subjects, using different contrasts and slice orientations.

The utility of the technique was demonstrated in online prospective self-gating. Several of the technical components of the approach can also be used in an offline retrospective self-gating, which might have certain benefits. For example, using the approach in FIG. 1 for CINE imaging inevitably will miss a fraction of the cardiac cycle as it needs to be used as a dedicated self-gating mode. This might be undesirable for CINE imaging and related volume and ejection fraction calculations. A retrospective offline self-gating might be more desirable. Nevertheless, the current approach suits well for non-cine type cardiac applications.

The PCA-based signal processing algorithm plays a key role in enabling online self-gating. A number of processing algorithms rely on a high order band-pass filter to suppress the non-cardiac signal component. Such high-order frequency filters are inherently slow and unsuitable for real time processing because of their group delay (37). In the proposed PCA algorithm, each self-gating sample is simply projected onto the principal component direction defined in the training phase. The PCA algorithm itself is causal with no processing delay, although the peak detection algorithm introduces a delay of 2 samples. As a result, it takes less than 10 ms for the sequence to detect the trigger and change mode accordingly, making the online prospective self-gating possible.

It should be noted that the self-gating triggers were delayed from the ECG triggers by an average of 228 ms for short-axis and 177 ms for vertical-long-axis. This is because: 1) there is an inherent delay between the electrical signal and the actual myocardial motion in which the electrical signal always comes first; 2) current self-gating trigger detection algorithm is based on finding the signal peak and thus tends to trigger on end-systole instead of end-diastole as the ECG R-wave based algorithm. A similar shift is also reported in other self-gating methods (38, 39).

The various methods and techniques described above provide a number of ways to carry out the invention. Of course, it is to be understood that not necessarily all objectives or advantages described may be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as may be taught or suggested herein. A variety of advantageous and disadvantageous alternatives are mentioned herein. It is to be understood that some preferred embodiments specifically include one, another, or several advantageous features, while others specifically exclude one, another, or several disadvantageous features, while still others specifically mitigate a present disadvantageous feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be mixed and matched by one of ordinary skill in this art to perform methods in accordance with principles described herein. Among the various elements, features, and steps some will be specifically included and others specifically excluded in diverse embodiments.

Although the invention has been disclosed in the context of certain embodiments and examples, it will be understood by those skilled in the art that the embodiments of the invention extend beyond the specifically disclosed embodiments to other alternative embodiments and/or uses and modifications and equivalents thereof.

In some embodiments, the terms “a” and “an” and “the” and similar references used in the context of describing a particular embodiment of the invention (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Preferred embodiments of this invention are described herein, including the best mode known to the inventors for carrying out the invention. Variations on those preferred embodiments will become apparent to those of ordinary skill in the art upon reading the foregoing description. It is contemplated that skilled artisans can employ such variations as appropriate, and the invention can be practiced otherwise than specifically described herein. Accordingly, many embodiments of this invention include all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that can be employed can be within the scope of the invention. Thus, by way of example, but not of limitation, alternative configurations of the present invention can be utilized in accordance with the teachings herein. Accordingly, embodiments of the present invention are not limited to that precisely as shown and described.

1. A method for synchronizing image data acquisition during Magnetic Resonance Imaging (MRI), comprising:
acquiring a self-gating dataset comprising a first plurality of subsets of self-gating data of the center k-space entire line, wherein the self-gating data are acquired separately from any imaging data, and wherein the first plurality of subsets of self-gating data is collected during the same cardiac cycle.

2. The method of claim 1, wherein the self-gating data is acquired using a plurality of radio frequency (RF) coil arrays.

3. The method of claim 1, wherein the first plurality of subsets of self-gating data is non-phase encoded.

4. The method of claim 1, wherein the self-gating dataset further comprises a second plurality of subsets of self-gating data.

5. The method of claim 1, wherein the first plurality and second plurality of subsets of self-gating data are collected during the same cardiac cycle.

6. The method of claim 1, wherein the first plurality and second plurality of subsets of self-gating data are collected during different cardiac cycles.

7. The method of claim 1, further comprising:
acquiring a training dataset comprising one or more subsets of training data, prior to the acquisition of the plurality of subsets of self-gating data.

8. The method of claim 7, wherein the training dataset is collected from a single cardiac cycle or a plurality of consecutive cardiac cycles.

9. The method of claim 7, wherein the training dataset is collected from a plurality of non-consecutive cardiac cycles.

10. The method of claim 1, wherein the training dataset is processed based on one or more training algorithms to produce a training result.

11. The method of claim 10, wherein the one or more training algorithms comprises principal component analysis, multilinear principal component analysis, a machine learning technique, independent component analysis (ICA), clustering analysis, analysis of variance (ANOVA) analysis, blind deconvolution, factor analysis, multilinear subspace learning, non-negative matrix factorization (NMF), nonlinear dimensionality reduction analysis, projection pursuit analysis, Varimax rotation analysis, and a combination thereof.

12. The method of claim 10, wherein the training result is selected from the group consisting of a principal component vector, a threshold for detecting a triggering event, an expected duration of a cardiac cycle, a parameter associated with an imaging device that is used for collecting the training dataset, and combinations thereof.

13. The method of claim 7, further comprising:
processing the one or more subsets of training data, based on one or more training algorithms.

14. The method of claim 10, wherein the plurality of subsets of self-gating data is processed based on the training result to detect the presence of a triggering event.

15. The method of claim 14, further comprising:
processing the plurality of subsets of self-gating data, based on the training result to detect the presence of the triggering event.

16. The method of claim 15, further comprising:
initiating image acquisition, upon detection of the onset of the triggering event.

17. The method of claim 16, wherein the triggering event is the onset of a heartbeat.

18. A data collection sequence for Magnetic Resonance Imaging (MRI) data acquisition, comprising:
a plurality of collection cycles, wherein at least one collection cycle in the plurality of collection cycles comprises:
a self-gating mode during which self-gating data is collected; and
an imaging mode during which image data is collected,
wherein the self-gating mode and the imaging mode in the at least one collection cycle do not overlap, and wherein non-phase encoded data of k-space center line is repeatedly acquired in the self-gating mode.

19. The data collection sequence of claim 18, wherein the at least one collection cycle corresponds to a cardiac cycle.

20. The data collection sequence of claim 18, wherein the self-gating data is non-phase encoded.

21. The data collection sequence of claim 18, wherein the self-gating data is acquired using a plurality of radio frequency (RF) coil arrays.

22. The data collection sequence of claim 19, wherein the training data is acquired using a plurality of radio frequency (RF) coil arrays.

23. The data collection sequence of claim 18, further comprising:
a training phase wherein training data is collected.

24. The data collection sequence of claim 22, wherein the training phase covers the duration of one or more cardiac cycles.
